(19) United States
(12) Patent Application Publication
(10) Pub. No.: US 2025/0340851 A1
(43) Pub. Date: Nov. 6, 2025

(54) ENZYME COMPOSITION WITH AT LEAST TWO DIFFERENT THERMOSTABLE POLYPEPTIDES HAVING TYPE II DNA METHYLTRANSFERASE ACTIVITY

(71) Applicant: BLUCON BIOTECH GMBH, Köln (DE)

(72) Inventors: Christian Zurek, Zwingenberg (DE); Rebecca Heger, Zwingenberg (DE); Steve Swinnen, Köln (DE); Marco Krämer; Jörg Mampel, Zwingenberg (DE)

(21) Appl. No.: 18/557,380

(22) PCT Filed: Apr. 28, 2022
(86) PCT No.: PCT/EP2022/061362

§ 371 Date: Oct. 26, 2023

(30) Foreign Application Priority Data
Apr. 28, 2021 (WO) PCT/EP2021/061199

(51) Int. Cl. C12N 9/10; C12N 1/20; C12N 15/75; C12R 1/01

(52) U.S. Cl. CPC C12N 9/1007; C12N 1/205; C12N 15/75; C12R 2001/01; C12Y 201/01072

(57) ABSTRACT

The invention relates to a novel enzyme composition comprising at least two different thermostable polypeptides having type II DNA methyltransferase activity as well as a restriction/modification system in particular for the transformation of microorganisms of the genus Caldicellulosiruptor, wherein said polypeptides methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

### CROSS REFERENCE TO RELATED APPLICATIONS

This is a US national phase application under 35 USC § 371 of international patent application no. PCT/EP2022/061362, filed Apr. 28, 2022, which itself claims priority to patent application no. PCT/EP2021/061199, filed Apr. 28, 2021. Each of the applications referred to in this paragraph are herein incorporated by reference in their entireties.

### REFERENCE TO SEQUENCE LISTING SUBMITTED ELECTRONICALLY

The official copy of the sequence listing is submitted electronically as an ASCII formatted sequence listing as file 18557380seqid created on Aug. 26, 2024, filed Aug. 26, 2024 and having a file size of 53 Kilobytes. The sequence listing forms part of the specification and is herein incorporated by reference in its entirety.

### FIELD OF THE DISCLOSURE

The present disclosure relates to a novel enzyme composition comprising at least two different thermostable polypeptides having type II DNA methyltransferase activity as well as a restriction/modification system in particular for the transformation of microorganisms of the genus Caldicellulosiruptor.

### BACKGROUND

Bacterial cells long have been known to contain restriction-modification systems that protect them from viral infection (see, for instance, Gingeras (1991)). A restriction-modification system generally operates through two complementing enzymatic activities, an endonucleolytic activity and a modification activity. The endonucleolytic activity involves recognition of a specific sequence in viral DNA and subsequent endonucleolytic cleavage across both strands of the DNA. The sequences, referred to as restriction recognition sites, usually have a length of only 4 to 8 base pairs, and are often palindromic. The generated fragments are degraded further by other endonucleases, thus successfully disposing of the foreign DNA that occurs in the cell.

The modification activity involves the same sequence recognition step followed by modification of a base in the sequence, which interferes with the endonucleolytic activity. Thus, host cell DNA modified by the endogenous modification enzyme, is protected from degradation by the endogenous endonuclease (also referred to as ‘restriction endonuclease’ or ‘restriction enzyme’) which destroys the unprotected DNA of infecting virus.

Restriction endonucleases belong to the class of enzymes called nucleases, which degrade or cut single or double stranded DNA. A restriction endonuclease acts by recognizing and binding to particular sequences of nucleotides (the ‘recognition sequence’ or ‘recognition site’) along the DNA molecule. Once bound, the endonuclease cleaves the molecule within or to one side of the recognition sequence. The location of cleavage may differ among various restriction endonucleases, though for any given endonuclease the position is fixed. Different restriction endonucleases have different affinity for recognition sequences. More than two hundred restriction endonucleases recognizing unique specificities have been identified among thousands of bacterial and archaeal species that have been examined to date.

A second component of bacterial and archaeal restriction-modification systems are the modification methylases (Roberts and Halford, in ‘Nucleases’, 2nd ed., Linn et al., ed.'s, p. 35-88 (1993)). These enzymes are complementary to restriction endonucleases and they provide the means by which bacteria are able to protect their own DNA and distinguish it from foreign, invading DNA. Modification methylases recognize and bind to the same recognition sequence as the corresponding restriction endonuclease, but instead of cleaving the DNA, they chemically modify one or other of the nucleotides within the sequence by the addition of a methyl group. Following methylation, the recognition sequence is no longer cleaved by the restriction endonuclease. The DNA of a bacterial cell is modified by virtue of the activity of its modification methylase, and is therefore insensitive to the presence of the endogenous restriction endonuclease. It is only unmodified and therefore identifiably foreign DNA that is sensitive to restriction endonuclease recognition and cleavage.

There are three major groups of DNA methyltransferases based on the position and the base that is modified (C5 cytosine methylases, N4 cytosine methylases, and N6 adenine methylases). N4 cytosine and N6 adenine methylases are amino-methyltransferases (Malone et al. J. Mol. Biol. 253:618-632 (1995)). When a restriction site on DNA is modified (methylated) by the methylase, it is resistant to cleavage by the cognate restriction endonuclease. Sometimes methylation by a non-cognate methylase can also confer DNA sites resistant to cleavage. For example, Dcm methylase modification of 5′ CCWGG 3′ (W=A or T) can also make the DNA resistant to PspGI cleavage. Another example is that CpM methylase can modify the CG dinucleotide and make the NotI site (5′ GCGGCCGC 3′) refractory to NotI cleavage (New England Biolabs' catalogue, 2000-01, page 220). Therefore, methylases can be used as a tool to modify certain DNA sequences and make them resistant to cleavage by restriction enzymes.

Although the restriction-modification system of a bacterium is an effective natural defence system against viruses, it forms a major obstruction for the introduction of foreign DNA in genetic engineering strategies. Hence, successful genetic engineering is only feasible after identifying and circumventing the bacterial restriction-modification system.

The restriction modification system of Caldicellulosiruptor bescii DSM 6725 has been identified before, as described in Chung et al., 2012 and filed by Westpheling et al., WO2013/184089 A1. The system is referred to as the CbeI/M.CbeI RM-system, named after the main restriction endonuclease responsible for DNA degradation. It has been shown that circumvention of the system by either methylation of the foreign DNA to be introduced or by deletion of the gene encoding for CbeI allowed for successful genetic engineering not only of Caldicellulosiruptor bescii DSM 6725 but also of Caldicellulosiruptor hydrothermalis (Chung et al., 2013).

Because purified restriction endonucleases and modification methylases are useful tools for creating recombinant molecules in the laboratory, there is a strong commercial interest to obtain novel enzymes and methods for a genetic modification of nucleic acids in other Caldicellulosiruptor species besides Caldicellulosiruptor bescii DSM 6725 and Caldicellulosiruptor hydrothermalis. Therefore, the availability of novel enzymes and methods would be highly advantageous.

### SUMMARY OF THE DISCLOSURE

The present invention relates to novel enzyme compositions comprising at least two different thermostable polypeptides having type II DNA methyltransferase activity, wherein
a) the first polypeptide is a thermostable polypeptide having type II DNA methyltransferase activity, wherein said polypeptide methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, and
b) the second polypeptide is a thermostable polypeptide having type II DNA methyltransferase activity, wherein said polypeptide methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In particular, the thermostable polypeptides comprised in an enzyme composition according to the present disclosure are N6 adenine methylases. Therefore, the methylation is in particular a N6-methyladenine modification.

In a further aspect, the present disclosure pertains to a polypeptide having restriction endonuclease activity, wherein the DNA recognition site of said polypeptide is 5′-GCATC-3′ and/or 3′-CGTAG-5′.

In a first aspect, the present disclosure pertains to polypeptide(s), in particular thermostable polypeptide(s), having type II DNA methyltransferase activity, wherein said polypeptide(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In a further aspect, the present disclosure pertains to a polypeptide, in particular a thermostable polypeptide, having restriction endonuclease activity, wherein the DNA recognition site of said polypeptide is 5′-GCATC-3′ and/or 3′-CGTAG-5′.

In particular, the polypeptides according to the present disclosure are isolated polypeptides. The term “isolated” describes any molecule separated from its natural source.

In a further aspect, the present disclosure pertains to an enzyme composition comprising one or at least two thermostable polypeptides according to the present disclosure.

Furthermore, the present disclosure pertains to a vector comprising a nucleic acid molecule according to the present disclosure and a host cell transformed, transduced or transfected with such a vector.

In particular, the present disclosure pertains to a restriction-modification system comprising a thermostable polypeptide having methyltransferase activity according to the present disclosure and a polypeptide having restriction endonuclease activity according to the present disclosure.

In a further aspect, the present disclosure pertains to a method for the in vitro methylation of DNA by using a thermostable polypeptide having methyltransferase activity according to the present disclosure or an enzyme composition according to the present disclosure.

In a further aspect, the present disclosure pertains a method for introducing an exogenous DNA molecule into a target bacterium, comprising steps of:
1) expression of a polypeptide, in particular of a thermostable polypeptide, having methyltransferase activity according to the present disclosure or of an enzyme composition according to the present disclosure in a recombinant microorganism;
2) introducing an exogenous target DNA molecule into said recombinant microorganism to obtain an exogenous target DNA molecule methylated by the polypeptide having methyltransferase activity; and
3) introducing said methylated exogenous target DNA molecule into the target bacterium.

Using the above-described methylation enzymes of the novel RM system for methylation of DNA allows introduction of foreign DNA into cells of Caldicellulosiruptor sp., in particular into the cells of the Caldicellulosiruptor sp. strain DIB 104C.

As a successful transformation process is a prerequisite for genetic and metabolic engineering of Caldicellulosiruptor sp. strain DIB 104C and clones derived thereof, implementation of the newly discovered RM system may allow strain improvement by molecular biology methods.

To provide a comprehensive disclosure without unduly lengthening the specification, the applicant hereby incorporates by reference each of the patents and patent applications cited herein.

### BRIEF DESCRIPTION OF THE DRAWINGS

FIG. 1 is a gel showing the digestion of genomic DNA from Caldicellulosiruptor sp. DIB 104C with methylation-sensitive restriction enzymes. Lane 1, DNA size standard; Lane 2, digestion with AluI; Lane 3, digestion with BamHI; Lane 4, digestion with BspEI; Lane 5, digestion with EcoRI; Lane 6, digestion with HaeIII; Lane 7, digestion with HhaI; Lane 8, digestion with HpaII; Lane 9, digestion with MboI; Lane 10, digestion with MspI; Lane 11, digestion with DpnI, Lane 12, digestion with SexAI; Lane 13, DNA size standard; Lane 14, negative control.

FIG. 2 shows the domain structure of the methyltransferase M.CbeI (picture taken from Chung et al., 2012). Predicted functional domains of M.CbeI and sequence alignments of conserved motifs of three M.CbeI homologues methyltransferases from different Caldicellulosiruptor species as well as of the M.CbeI homologues methyltransferase DmtB from Anabaena variabilis.

FIG. 3 is a tblastn analysis applying the Caldicellulosiruptor sp. BluCon085 genome sequence and the M.CbeI protein sequence. The result for the best-hit protein is shown.

FIG. 4 is a sequence alignment of the ORF03237 protein sequence of Caldicellulosiruptor sp. BluCon085 against methyltransferases of several species of the genus Caldicellulosiruptor.

FIG. 5 shows the identification of genes encoding a probable type IIS RM-system in Caldicellulosiruptor sp. BluCon085 as predicted for Caldicellulosiruptor saccharolyticus DSM8903 in REBASE. The type IIS RM-system of Caldicellulosiruptor sp. BluCon085 consists of an operon comprising of one restriction enzyme (encoded by DOCILOBI_02329) and two methyltransferases (encoded by DOCILOBI_02330 and DOCILOBI_02331, respectively).

FIG. 6 is a violin plot presentation of methylated base pairs including the sequence motif 5′-GATGC-3′. The motif “AT” is methylated.

FIG. 7 is a violin plot presentation of methylated base pairs including the sequence motif 5′-GCATC-3′. The motif “AT” is methylated.

FIG. 8 is a violin plot presentation of methylated base pairs including the sequence motif 5′-GATC-3′. The bases “AT” are most likely methylated.

FIG. 9 shows the predicted restriction and modification systems in Caldicellulosiruptor saccharolyticus DSM 8903 according to the REBASE database (http://rebase.neb.com/rebase/rebase.html).

FIG. 10 shows the results of a blastp analysis for closely related restriction enzymes and their specificities to CsaDORF2754P from Caldicellulosiruptor saccharolyticus DSM8903 in REBASE.

FIG. 11 shows the predicted recognition and methylation sites of the annotated type IIS RM-system in Caldicellulosiruptor sp. DIB 104C and Caldicellulosiruptor sp. BluCon085.

FIG. 12 are two gels showing the digestion of genomic DNA from Caldicellulosiruptor sp. BluCon085 (methylated) (left) and plasmid DNA pF25-5br (unmethylated) (right) with various methylation-sensitive restriction enzymes. Lane M: DNA size standard; Lane A: AlwI; Lane P: PleI; Lane S: SfaNI; Lane D: DpnI; Lane C: Control, without restriction enzyme.

FIG. 13 is a gel showing the incubation of a methylated and non-methylated DNA fragment with cell lysate from Caldicellulosiruptor sp. BluCon085. Lane M: DNA size standard; Lane 1: without cell lysate; Lane 2: 14% (v/v) cell lysate; Lane 3: 29% (v/v) cell lysate; Lane 4: 43% (v/v) cell lysate.

FIG. 14 is a schematic representation of the intact pyrE locus of Caldicellulosiruptor sp. DIB 104C with the targeted BglII introduction site. The deletion of 114 bp in strain Caldicellulosiruptor sp. DIB 104C pyrE M62 is also shown.

FIG. 15 is a gel showing the verification of DNA methylation by SfaNI hydrolysis. Lane M: DNA size standard; Lane 1: pyrE-PCR amplicon; Lane 2; pyrE-PCR amplicon, methylated; Lane 3: pF25-6brBglII; Lane 4: pF25-6brBglII, methylated.

FIG. 16 is a gel showing the BglII hydrolysis of PCR amplicons obtained with primer pair BLU001 plus BLU002. Lane M: DNA size standard; Lanes #1-7: Uracil-protrophic Caldicellulosiruptor sp. DIB 104C pyrE M62 clones; Lane M62: Caldicellulosiruptor sp. DIB 104C pyrE M62; Lane C15: Caldicellulosiruptor sp. DIB 104C; Lane P: Plasmid pF25-6brBglII.

FIG. 17 is a gel showing BglII hydrolysis of PCR amplicon obtained with primer pair primers 054_pyrEup_f and 055_pyrEdw_r. Lane M: DNA size standard; Lanes #1-7: Uracil-protrophic Caldicellulosiruptor sp. DIB 104C pyrE M62 clones; Lane M62: Caldicellulosiruptor sp. DIB 104C pyrE M62; Lane C15: Caldicellulosiruptor sp. DIB 104C; Lane P: Plasmid pF25-6brBglII.

FIG. 18 shows an alignment of the sequences from the fragments generated by PCR with primers 054_pyrEup_f and 055_pyrEdw_r on genomic DNA from DIB 104C pyrE M62 clones #1 to #3 compared to the pyrE wild-type.

FIG. 19 is the sequence of plasmid pF25-5br (SEQ ID NO: 1).

FIG. 20 is the sequence of plasmid pTrc-EcMt1ORF02330 with gene DOCILOBI_02330 codon-optimized for expression in E. coli (SEQ ID NO: 2).

FIG. 21 is the sequence of plasmid pTrc-EcMt2ORF02331 with gene DOCILOBI_02331 codon-optimized for expression in E. coli (SEQ ID NO: 3).

FIG. 22 is the sequence of plasmid pF25-6brBglII (SEQ ID NO: 4).

FIG. 23 shows the sequence of gene DOCILOBI_02330 encoding for methyltransferase 1. A) Gene in plasmid pTrc-EcMt1ORF02330 codon-optimized for expression in E. coli (SEQ ID NO: 5); B) Amino acid sequence of the translation product of SEQ ID NO: 5 (SEQ ID NO: 6).

FIG. 24 shows the sequence of gene DOCILOBI_02330 encoding for methyltransferase 1. A) Gene in Caldicellulosiruptor sp. DIB 104C (SEQ ID NO: 7); B) Amino acid sequence of the translation product of SEQ ID NO.7 (SEQ ID NO: 8).

FIG. 25 shows the sequence of gene DOCILOBI_02331 encoding for methyltransferase 2. A) Gene in plasmid pTrc-EcMt2ORF02331 codon-optimized for expression in E. coli (SEQ ID NO. 9); B) Amino acid sequence of the translation product of SEQ ID NO. 9 (SEQ ID NO. 10).

FIG. 26 shows the sequence of gene DOCILOBI_02331 encoding for methyltransferase 2. A) Gene in Caldicellulosiruptor sp. DIB 104C (SEQ ID NO: 11); B) Amino acid sequence of the translation product of SEQ ID NO:11 (SEQ ID NO: 12).

FIG. 27 shows the sequence of gene DOCILOBI_02329 ((SEQ ID NO: 12) encoding for an embodiment of a restriction enzyme type II of the restriction-modification system according to the present description.

### DETAILED DESCRIPTION OF THIS DISCLOSURE

The present disclosure relates to a novel restriction-modification system, in particular in microorganisms of the genus Caldicellulosiruptor, comprising at least two different methyltransferases having thermophilic activity profiles, and a restriction enzyme. The methyltransferases of the present description methylate at least one inner adenine residue in the DNA recognition sequence 5′-GCATC-3′ and/or in the complement DNA recognition site 3′-CGTAG-5′.

Furthermore, the polypeptide having restriction endonuclease activity (restriction enzyme) comprised in the restriction-modification system according to the present description has a DNA recognition site of 5′-GCATC-3′ and/or 3′-CGTAG-5′.

The terms “polypeptide”, “peptide”, or “protein” are used interchangeably herein to designate a linear series of amino acid residues connected one to the other by peptide bonds between the alpha-amino and carboxyl groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”.

Thus, the description provides, in various aspects, isolated thermostable polypeptide(s) having type II DNA methyltransferase activity or biologically active fragments/variants thereof (methyltransferase) and polypeptide(s) having restriction endonuclease activity (restriction endonuclease); isolated polynucleotides that encode the polypeptides or biologically active fragments thereof, including expression vectors that include such polynucleotide sequences; methods of digesting DNA using said restriction endonuclease; methods of treating a DNA molecule using a methyltransferase according to the present disclosure and/or a restriction-modification system according to the present disclosure; and methods of transforming a Caldicellulosiruptor cell. Because members of the genus Caldicellulosiruptor possess certain biology properties of potential commercial value (e.g., biomass conversion), the ability to genetically manipulate these organisms can assist in metabolically engineering members of this genus for, for example, their use in consolidated bioprocessing that produces one or more biofuels and/or one or more bio products, in particular lactate.

In particular, the isolated thermostable polypeptide(s) having type II DNA methyltransferase activity or biologically active fragments/variants thereof (methyltransferase) and polypeptide(s) having restriction endonuclease activity (restriction endonuclease) are codon-optimized for the expression in E. coli.

Thus, certain aspects of the description can be used to overcome restriction that may assist methods of DNA transformation of Caldicellulosiruptor species using DNA from, for example, homologous and/or heterologous sources. Moreover, these aspects may be generalized to permit transformation of other thermophilic and/or hyperthermophilic microbes.

An advantage property of members of the genus Caldicellulosiruptor is the high temperature tolerance, which is higher than 70 degrees centigrade for fermentative lactic acid production, which is a higher temperature tolerance compared to the members of the family of the Lactobacillaceae and members of the family of the Bacillaceae. All members of the genus Caldicellulosiruptor could be used for the conversion processes.

Using the above-described methylation enzymes of the novel RM system for methylation of DNA allows introduction of foreign DNA into cells/microorganisms, in particular into Caldicellulosiruptor sp. strains like DIB 104C.

As a successful transformation process is a prerequisite for genetic and metabolic engineering of microorganisms, in particular of Caldicellulosiruptor sp. strains like DIB 104C and clones derived thereof, implementation of the newly discovered RM system may allow strain improvement by molecular biology methods.

A further method of the present disclosure pertains to the knockout of gene(s) encoding the restriction endonuclease according to the present disclosure in a microorganism e.g., by mutagenesis. Then, foreign (exogeneous) DNA may be introduced into the microorganism like the Caldicellulosiruptor sp. strain DIB 104C.

Furthermore, DNA might be cloned in suitable E. coli strains or other suitable recombinant strains exhibiting a methylation pattern that is identical to Caldicellulosiruptor sp. strain DIB 104C. This methylated DNA will be compatible with the RM system of the present disclosure, in particular of the RM system of Caldicellulosiruptor sp. strain DIB 104C.

The application of this technology has the potential to improve microorganisms, in particular Caldicellulosiruptor sp. strains, in particular Caldicellulosiruptor sp. strain DIB 104C for the production of carbon-based chemicals like lactate rendering the process more economically feasible. In particular, these microorganisms are extremely thermophilic and show broad substrate specificities and high natural production of lactic acid. Moreover, lactic acid fermentation at high temperatures, for example over 70 degrees centigrade has many advantages over mesophilic fermentation. One advantage of thermophilic fermentation is the minimization of the problem of contamination in batch cultures, fed-batch cultures or continuous cultures, since only a few microorganisms are able to grow at such high temperatures in un-detoxified starch biomass material. Another aspect of fermentations at high temperatures is that viscosity of the culture is dramatically reduced decreasing the required electric energy input for stirring. Additionally, energy for cooling of the process is not necessary.

The polypeptides according to the present disclosure are preferably thermostable, i.e., they are enzymatically active at high temperatures even at or above 70° C., in particular between 70° C. and 85° C.

The polypeptide having restriction endonuclease activity according to the present disclosure refers to a polypeptide that cleaves DNA and the DNA recognition site of said polypeptide is 5′-GCATC-3′ and/or 3′-CGTAG-5′. This restriction endonuclease may be a polypeptide encoded by SEQ ID NO. 13, or a biologically active fragment of such a polypeptide. Biological activity, in the context of the restriction endonuclease refers to the ability to digest DNA specifically at a 5′-GCATC-3′ and/or 3′-CGTAG-5′ recognition site at a temperature from 35° C. to 85° C., in particular at a temperature between 70° C. and 85° C.

As used herein the term “endonuclease” refers to an enzyme capable of causing a single or double-stranded break in a DNA molecule. Type II restriction endonucleases are a class of enzymes that occur naturally in bacteria and in some viruses. When they are purified away from other bacterial/viral proteins, restriction endonucleases can be used in the laboratory to cleave DNA molecules into small fragments for molecular cloning and gene characterization. Restriction endonucleases recognize and bind particular sequences of nucleotides (the recognition sequence) along the DNA molecules. Once bound, they cleave the molecule within (e. g. BamHI), to one side of (e. g. SapI), or to both sides of (e. g. TspRI) the recognition sequence. Different restriction endonucleases have affinity for different recognition sequences.

The polypeptide having restriction endonuclease activity according to the present disclosure are in particular type IIS restriction enzymes. When type IIS enzymes bind to DNA, the catalytic domain is positioned to one side of, and several bases away from, the sequence bound by the recognition domain, and so cleavage is ‘shifted’ to one side of the sequence. Type IIS enzymes generally bind to DNA as monomers and recognize asymmetric DNA sequences. They cleave outside of this sequence, within one to two turns of the DNA. By convention, the recognition sequence is written in the orientation in which cleavage occurs downstream, to the right of the sequence. Cleavage often produces staggered ends of two or four bases. The exact positions of cleavage are indicated by the number of bases away from the recognition sequence in each strand. For example, the polypeptide having restriction endonuclease activity according to the present disclosure recognizes the asymmetric sequence 5′-GCATC-3′ in duplex DNA and cleaves this strand downstream to the recognition site and produces 5′-overhanging ends.

Thus, according to the present disclosure, there is provided a restriction endonuclease, which is characterized by the asymmetric recognition sequence:

5′-GCATC-3′
3′-CGTAG-5′

The new Class II restriction endonuclease according to the present invention has an average temperature optimum of 35° C. to 85° C., in particular between 70° C. and 85° C. and a pH optimum between pH 7.2 and pH 8.0, in particular at a concentration of a monovalent cation at 70 mmol/l potassium acetate.

In an advantageous embodiment, the present disclosure relates to a polypeptide having restriction endonuclease activity, wherein the DNA recognition site of said polypeptide is 5′-GCATC-3′ and/or 3′-CGTAG-5′. In particular, said polypeptide is encoded by the nucleic acid sequence of SEQ ID NO. 13 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 13, wherein the DNA recognition site of said variant(s) is 5′-GCATC-3′ and/or 3′-CGTAG-5′.

The term “variant” means that the amino acid sequence has been modified but retains the same functional characteristics, in particular the restriction endonuclease activity and in view of the restriction endonucleases according to the present disclosure, wherein the DNA recognition site is still 5′-GCATC-3′ and/or 3′-CGTAG-5. A further characteristic could be the thermostability of the restriction endonuclease. In view of the polypeptide(s) having type II DNA methyltransferase activity according to the present disclosure, the functional characteristics are the thermostability and that said methyltransferase methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

A variant has a sequence identity of at least 70% or preferably at least 80%, 85%, 90%, 95%, 97% or 99% to the parent amino acid sequence. The term “variant” refers further to a polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For example: 4-hydroxyproline may be substituted for proline; 5-hydroxylysine may be substituted for lysine; 3methylhistidine may be substituted for histidine; homoserine may be substituted for serine; and ornithine may be substituted for lysine.

As mentioned above, a second component of the restriction-modification systems according to the present disclosure are methylases (methyltransferases). These enzymes co-exist with restriction endonucleases and they provide the means by which bacteria are able to protect their own DNA and distinguish it from foreign DNA. Modification methylases recognize and bind to the same recognition sequence as the corresponding restriction endonuclease, but instead of cleaving the DNA, they chemically modify one particular nucleotide within the sequence by the addition of a methyl group (C5 methyl cytosine, N4 methyl cytosine, or N6 methyl adenine). Following methylation, the recognition sequence is no longer cleaved by the cognate restriction endonuclease. The DNA of a bacterial cell is always fully modified by the activity of its modification methylase. It is therefore completely insensitive to the presence of the endogenous restriction endonuclease. Only unmodified, and therefore identifiably foreign DNA, is sensitive to restriction endonuclease recognition and cleavage. During and after DNA replication, usually the hemi-methylated DNA (DNA methylated on one strand) is also resistant to the cognate restriction digestion.

Therefore, the present disclosure pertains to a polypeptide having type II DNA methyltransferase activity and refers to a polypeptide that, when incubated with DNA at a temperature from 35° C. to 85° C., in particular between 70° C. and 85° C. methylates an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′. In particular, the methylation is a N6-methyladenine modification and the thermostable polypeptide is therefore a N6 adenine methylase.

In advantageous embodiments, the methylase according to the present disclosure is selected from the group consisting of a polypeptide having type II DNA methyltransferase activity comprising the amino acid sequence of SEQ ID NO: 6 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 6, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, a polypeptide having type II DNA methyltransferase activity comprising the amino acid sequence of SEQ ID NO: 8 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, a polypeptide having type II DNA methyltransferase activity comprising the amino acid sequence of SEQ ID NO: 10 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 10, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′ and a polypeptide having type II DNA methyltransferase activity comprising the amino acid sequence of SEQ ID NO: 12 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′ or combinations thereof.

“Methylase” and “methyltransferase” are synonymous as used herein and may be used interchangeably. The term “and/or” means one or all of the listed elements or a combination of any two or more of the listed elements. For example, Jurkowska R Z, Ceccaldi A, Zhang Y, Arimondo P B, Jeltsch A. DNA methyltransferase assays. Methods Mol Biol. 2011; 791:157-77. doi: 10.1007/978-1-61779-316-5_13. PMID: 21913079 describes different DNA methyltransferase assays. Furthermore, Wood R J, McKelvie J C, Maynard-Smith M D, Roach P L. A real-time assay for CpG-specific cytosine-C5 methyltransferase activity. Nucleic Acids Res. 2010 May; 38(9):e107. doi: 10.1093/nar/gkq047. Epub 2010 Feb. 5. PMID: 20139415; PMCID: PMC2875032 pertains to a real-time assay for CpG-specific cytosine-C5 methyltransferase activity. Further, Poh W J, Wee C P, Gao Z. DNA Methyltransferase Activity Assays: Advances and Challenges. Theranostics. 2016; 6(3):369-391. Published 2016 Jan. 6. doi:10.7150/thno.13438 discloses further DNA Methyltransferase Activity Assays like Radio DNA MTase assays, Colourimetric DNA MTase activity assays, Fluorescent DNA MTase activity assays, Chemiluminescent/bioluminescent DNA MTase activity assays, Electrochemical DNA MTase activity assays, ECL DNA MTase activity assays and other assays, in particular the assay shown in the examples of the present disclosure.

In the present disclosure, the occurrence of methyltransferase activity was shown by the verification of the methylated DNA product, which was not digested by the suitable endonuclease SfaNI. The methylated DNA product was transformed into Caldicellulosiruptor DIB 104C, which resulted in successful transformation of the strain, while introduction of the non-methylated DNA did not result in transformation. A similar approach has been carried out before by Chung et al. (2012). However, the authors used another endonuclease, HaeIII, the isochizomer of CbeI, which is the endonuclease of the restriction-modification system of Caldicellulosiruptor bescii.

The present disclosure pertains in particular to a method for the methylation of DNA comprising expressing at least a polypeptide having type II DNA methyltransferase activity according to the present disclosure or at least two different polypeptides having type II DNA methyltransferase activity according to the present disclosure in a microorganism, in particular a recombinant microorganism, wherein an exogenous target DNA molecule (foreign DNA) is methylated in said microorganism. After this, the methylated target DNA may be introduced in a target bacterium according to the present disclosure.

As mentioned above, the present disclosure pertains further to nucleic acid molecules encoding a DNA methyltransferase and/or restriction endonuclease. In particular, the nucleic acid molecule comprises a nucleic acid sequence selected from the group consisting of SEQ ID NO.5, SEQ ID NO. 7, SEQ ID NO. 9 and SEQ ID NO. 11 or variants thereof, wherein the nucleic acid sequence of said variants comprising at least a minimum percentage sequence identity of at least 85%, at least 90%, at least 93%, at least 96%, at least 97%, at least 98% or at least 99% to the nucleic acid sequence of SEQ ID NO.5, SEQ ID NO. 7, SEQ ID NO. 9 and SEQ ID NO. 11, and wherein said variant(s) encodes a thermostable polypeptide that methylates an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In a further embodiment, the nucleic acid molecule comprises the nucleic acid sequence of SEQ ID NO. 13 or variants thereof, wherein the nucleic acid sequence of said variants comprising at least a minimum percentage sequence identity of at least 85%, at least 90%, at least 93%, at least 96%, at least 97%, at least 98% or at least 99% to the nucleic acid sequence of SEQ ID NO.5, SEQ ID NO. 7, SEQ ID NO. 9 and SEQ ID NO. 11, and wherein said variant(s) encodes a polypeptide having restriction endonuclease activity, wherein the DNA recognition site of said polypeptide is 5′-GCATC-3′ and/or 3′-CGTAG-5′.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA) and the term “deoxyribonucleic acid” (DNA) is inclusive of cDNA and genomic DNA and DNA-RNA hybrids. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides or peptides.

As used herein, the phrase “encoding nucleic acid”, “coding sequence”, “encoding sequence”, “structural nucleotide sequence” or “structural nucleic acid molecule” refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

The term “complementary” as used herein refers to a relationship between two nucleic acid sequences. One nucleic acid sequence is complementary to a second nucleic acid sequence if it is capable of forming a duplex with the second nucleic acid, wherein each residue of the duplex forms a guanosine-cytidine (G-C) or adenosine-thymidine (A-T) base pair or an equivalent base pair. Equivalent base pairs can include nucleoside or nucleotide analogues other than guanosine, cytidine, adenosine, or thymidine.

As mentioned above, the present disclosure pertains further to a vector comprising a nucleic acid molecule according to the present description. In particular, the vector comprises the sequence of a polypeptide having methyltransferase activity according to the present disclosure, in particular SEQ ID NO. 2 and/or SEQ ID NO 3.

Furthermore, the present disclosure pertains to a host cell transformed, transduced or transfected with a vector according to the present description. The term “host cell(s)” refers to cell(s), which may be used for the methylation of an exogenous target DNA in accordance with the present disclosure. A host cell, according to the present disclosure may be, but is not limited to, prokaryotic cells, eukaryotic cells, archeobacteria, bacterial cells, insect cells, yeast, mammal cells, and/or plant cells. Bacteria envisioned as host cells can be either gram-negative or gram-positive, e.g. Escherichia coli, Erwinia sp., Klebsiella sp., Lactobacillus sp. or Bacillus subtilis. Typical yeast host cells are selected from the group consisting of Saccharomyces cerevisiae, Hansenula polymorpha and Pichia pastoris.

Further, a host cell may be characterized in that a polypeptide having restriction endonuclease activity according to the present disclosure is inhibited by an inhibitor in the host cell and/or the gene encoding said polypeptide is knocked-out in the host cell, wherein said inhibitor inhibits the expression of said polypeptide and/or binds to a protein product of a gene coding said polypeptide.

In an advantageous embodiment, at least two different of the above-mentioned methylases are comprised in a composition (enzyme composition). This composition according to the present disclosure may be used for the methylation of DNA.

In an advantageous embodiment, the enzyme composition according to the present disclosure of one of the polypeptides comprise the amino acid sequence of SEQ ID NO: 6 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 6, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In an advantageous embodiment, the enzyme composition according to the present disclosure of one of the polypeptides comprise the amino acid sequence of SEQ ID NO: 8 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In an advantageous embodiment, the enzyme composition according to the present disclosure of one of the polypeptides comprise the amino acid sequence of SEQ ID NO: 10 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 10, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In an advantageous embodiment, the enzyme composition according to the present disclosure of one of the polypeptides comprise the amino acid sequence of SEQ ID NO: 12 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In an advantageous embodiment, the enzyme composition according to the present disclosure of one of the polypeptides comprise the amino acid sequence of SEQ ID NO: 6 or SEQ ID NO: 8, or variants of SEQ ID NO: 6 or SEQ ID NO: 8, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 6 or SEQ ID NO: 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, and the second thermostable polypeptide comprises the amino acid sequence of SEQ ID NO: 10 or SEQ ID NO: 12, or variants of SEQ ID NO: 10 or SEQ ID NO: 12, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 10 or SEQ ID NO: 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

As mentioned above, the present disclosure pertains to a restriction-modification system comprising a polypeptide having methyltransferase activity according to the present disclosure and a polypeptide having restriction endonuclease activity according to the present disclosure. In particular, the restriction-modification system comprises an enzyme composition according to the present disclosure and a polypeptide having restriction endonuclease activity according the present disclosure. In an advantageous embodiment, the restriction-modification system comprises a first thermostable polypeptide comprising the amino acid sequence of SEQ ID NO: 6 or SEQ ID NO: 8, or variants of SEQ ID NO: 6 or SEQ ID NO: 8, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 6 or SEQ ID NO: 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, and a second thermostable polypeptide comprising the amino acid sequence of SEQ ID NO: 10 or SEQ ID NO: 12, or variants of SEQ ID NO: 10 or SEQ ID NO: 12, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 10 or SEQ ID NO: 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′; and a polypeptide having restriction endonuclease activity, wherein the DNA recognition site of said polypeptide is 5′-GCATC-3′ and/or 3′-CGTAG-5′, wherein said polypeptide is encoded by the nucleic acid sequence of SEQ ID NO: 13 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 13, wherein the DNA recognition site of said variant(s) is 5′-GCATC-3′ and/or 3′-CGTAG-5′.

As mentioned above, the present disclosure pertains to a method for the in vitro methylation of DNA by using a polypeptide according to the present disclosure or an enzyme composition according to the present disclosure.

Furthermore, the present disclosure relates to a method for introducing an exogenous DNA molecule into a target bacterium, comprising steps of:
1) expression of a polypeptide having methyltransferase activity according to the present disclosure or of an enzyme composition according to the present disclosure in a microorganism, in particular a recombinant microorganism;
2) introducing an exogenous target DNA molecule into said microorganism to obtain an exogenous target DNA molecule methylated by said polypeptide having methyltransferase activity; and
3) introducing said methylated exogenous target DNA molecule into the target bacterium.

The phrase “recombinant” and like terms refers to a nucleic acid, protein or microorganism which contains portions of different individuals, different species, or different genera that have been joined together. Typically, this is done using techniques of recombinant DNA, such that a composite nucleic acid is formed. The composite nucleic acid can be used to make a composite protein, for example. It can be used to make a fusion protein. It can be used to transform a microbe, which maintains and replicates the composite nucleic acid and optionally expresses a protein, optionally a composite protein.

The term “a bacterium” as used herein may further refer to only one unicellular organism as well as to numerous single unicellular organisms. For example, the term “a bacterium of the genus Caldicellulosiruptor” may refer to one single Caldicellulosiruptor bacterial cell of the genus Caldicellulosiruptor as well as to multiple bacterial cells of the genus Caldicellulosiruptor.

The terms “a strain of the genus Caldicellulosiruptor” and “a Caldicellulosiruptor cell” are used synonymously herein. In general, the term “a microorganism” refers to numerous cells. In particular, said term refers to at least 10³ cells, preferably at least 10⁴ cells, at least 10⁵ or at least 10⁶ cells.

As used in the present disclosure, “cell”, “cell line”, and “cell culture” can be used interchangeably and all such designations include progeny. It is also understood that all progeny may not be precisely identical in DNA content, due to deliberate or inadvertent mutations. Mutant progeny that have the same functionality as screened for in the originally transformed cell are included. As used herein, the phrase “coding sequence”, “encoding sequence”, “structural nucleotide sequence” or “structural nucleic acid molecule” refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

The target bacterium according to the present disclosure is in particular a bacterium of the species Caldicellulosiruptor saccharolyticus, Caldicellulosiruptor changbaiensis, Caldicellulosiruptor naganoensis and the species or strain Caldicellulosiruptor sp. E32, in particular an isolated bacterium of the genus Caldicellulosiruptor sp., wherein the bacterium may be selected from the group consisting of Caldicellulosiruptor sp. BluConL70 having the DSMZ Accession number 33496, Caldicellulosiruptor sp. BluConL60 having the DSMZ Accession number 33252, Caldicellulosiruptor sp. BluCon085 having the DSMZ Accession number 33485, Caldicellulosiruptor sp. BluCon052 having the DSMZ Accession number 33470, Caldicellulosiruptor sp. BluCon006 having the DSMZ Accession number 33095, Caldicellulosiruptor sp. BluCon014 having the DSMZ Accession number 33096 and Caldicellulosiruptor sp. BluCon016 having the DSMZ Accession number 33097, microorganism derived therefrom, progenies or mutants thereof, wherein the mutants thereof retaining the properties of BluConL70, BluConL60, BluCon085, BluCon052, BluCon006, BluCon014 and/or BluCon016.

In some advantageous embodiments, the target bacterium is an isolated bacterium of the genus Caldicellulosiruptor sp., wherein the bacterium is a microorganism of the genus Caldicellulosiruptor which is selected from the group consisting of Caldicellulosiruptor sp. DIB 041C (DSMZ Accession number 25771), Caldicellulosiruptor sp. DIB 004C (DSMZ Accession number 25177), Caldicellulosiruptor sp. DIB 101C (DSMZ Accession number 25178), Caldicellulosiruptor sp. DIB 103C (DSMZ Accession number 25773), Caldicellulosiruptor sp. DIB 107C (DSMZ Accession number 25775), Caldicellulosiruptor sp. DIB 087C (DSMZ Accession number 25772), Caldicellulosiruptor sp. DIB 104C (DSMZ Accession number 25774), Caldicellulosiruptor sp. BluCon006 (DSMZ Accession number 33095), Caldicellulosiruptor sp. BluCon014 (DSMZ Accession number 33096), Caldicellulosiruptor sp. BluCon016 (DSMZ Accession number 33097) and Caldicellulosiruptor sp. BluConL60 (DSMZ Accession number 33252).

In some embodiments, the target bacterium is Caldicellulosiruptor sp. DIB 104C (DSMZ Accession number 25774) or Caldicellulosiruptor sp. BluCon085 (DSMZ Accession number 33485).

TABLE 1
Strains of Caldicellulosiruptor used as target bacteria

| | | | DSMZ | | |
|---|---|---|---|---|---|
| | | | accession | Deposition | |
| Genus | Species | Name | number | date | Depositor |
| Caldicellulosiruptor | sp. | DIB004C | DSM 25177 | Sep. 15, 2011 | DIREVO Industrial |
| Caldicellulosiruptor | sp. | DIB041C | DSM 25771 | Mar. 15, 2012 | Biotechnology GmbH |
| Caldicellulosiruptor | sp. | DIB087C | DSM 25772 | Mar. 15, 2012 | Nattermannallee 1 |
| Caldicellulosiruptor | sp. | DIB101C | DSM 25178 | Sep. 15, 2011 | 50829 Cologne (DE) |
| Caldicellulosiruptor | sp. | DIB103C | DSM 25773 | Mar. 15, 2012 | |
| Caldicellulosiruptor | sp. | DIB104C | DSM 25774 | Mar. 15, 2012 | |
| Caldicellulosiruptor | sp. | DIB107C | DSM 25775 | Mar. 15, 2012 | |
| Caldicellulosiruptor | sp. | BluCon006 | DSM 33095 | Apr. 9, 2019 | BluCon Biotech GmbH |
| Caldicellulosiruptor | sp. | BluCon014 | DSM 33096 | Apr. 9, 2019 | Nattermannallee 1 |
| Caldicellulosiruptor | sp. | BluCon016 | DSM 33097 | Apr. 9, 2019 | 50829 Cologne |
| Caldicellulosiruptor | sp. | BluConL60 | DSM 33252 | Aug. 29, 2019 | |
| Caldicellulosiruptor | sp | BluConL70 | DSM 33496 | Mar. 20, 2020 | |
| Caldicellulosiruptor | sp | BluCon085 | DSM 33485 | Mar. 10, 2020 | |
| Caldicellulosiruptor | sp | BluCon0052 | DSM 33470 | Mar. 10, 2020 | |
| Caldicellulosiruptor | sp | DIB 104C | DSM 33675 | Oct. 22, 2020 | |
| | | pyrE M62 | | | |

The strains listed in Table 1 have been deposited in accordance with the terms of the Budapest Treaty on Sep. 15, 2011 with DSMZ—Deutsche Sammlung von Mikroorganismen und Zellkulturen GmbH, Inhoffenstraße 7B3, 38124 Braunschweig, Germany, under the respectively indicated DSMZ accession numbers and deposition dates.

In a preferred embodiment, the Caldicellulosiruptor sp. bacterium is
a) Caldicellulosiruptor sp. strain BluConL60 that was deposited on Aug. 29, 2019 under the accession number DSM 33252 according to the requirements of the Budapest Treaty at the Deutsche Sammlung von Mikroorganismen und Zellkulturen (DSMZ), Inhoffenstraße 7B3, 38124 Braunschweig (DE) by BluCon Biotech GmbH, Nattermannallee 1, 50829 Cologne (DE),
b) a microorganism derived from Caldicellulosiruptor sp. BluConL60 or
c) a Caldicellulosiruptor sp. BluConL60 mutant retaining the properties of BluConL60.

All strains and mutants thereof shown in Table 1 belong to the genus Caldicellulosiruptor and are strictly anaerobic, non-spore forming, non-motile, gram-positive bacteria. Cells are straight rods 0.4-0.5 μm by 2.0-4.0 μm, occurring both singly and in pairs.

The present disclosure pertains further to a method for the methylation of DNA comprising treating a DNA molecule in vitro or in vivo with a polypeptide having type II DNA methyltransferase activity according to the present disclosure or an enzyme composition comprising at least two polypeptides having type II DNA methyltransferase activity according to the present disclosure. In particular, in said method for the methylation of DNA, the DNA molecules are treated with an enzyme composition comprising at least two different thermostable polypeptide(s) having type II DNA methyltransferase activity, wherein the first thermostable polypeptide comprises—
the amino acid sequence of SEQ ID NO: 6 or SEQ ID NO: 8, or variants of SEQ ID NO: 6 or SEQ ID NO: 8, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 6 or SEQ ID NO: 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, and the second thermostable polypeptide comprises
the amino acid sequence of SEQ ID NO: 10 or SEQ ID NO: 12, or variants of SEQ ID NO: 10 or SEQ ID NO: 12, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 10 or SEQ ID NO: 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

The present disclosure pertains further to a method for introducing an exogenous DNA molecule into a target bacterium, comprising steps of:
1) co-expressing, in a microorganism, in particular in E. coli, at least two different DNA-methyltransferase-encoding genes encoding the thermostable polypeptides having type II DNA methyltransferase activity according to any one of claims 1 to 6 to obtain a recombinant bacterium A;
2) introducing an exogenous plasmid DNA molecule into said recombinant bacterium A for in vivo modification to obtain a methylated exogenous plasmid DNA molecule; and
3) introducing said methylated exogenous plasmid DNA molecule into said target bacterium.

In some advantageous embodiment one of the thermostable polypeptides (the first thermostable polypeptide) comprises—
the amino acid sequence of SEQ ID NO: 6 or SEQ ID NO: 8, or variants of SEQ ID NO: 6 or SEQ ID NO: 8, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 6 or SEQ ID NO: 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, and the second thermostable polypeptide comprises
the amino acid sequence of SEQ ID NO: 10 or SEQ ID NO: 12, or variants of SEQ ID NO: 10 or SEQ ID NO: 12, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 10 or SEQ ID NO: 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In some advantageous embodiments, the DNA-methyltransferase-encoding genes comprise a nucleic acid sequence selected from the group consisting of SEQ ID NO.5, SEQ ID NO. 7, SEQ ID NO. 9 and SEQ ID NO. 11 or variants thereof, wherein the nucleic acid sequence of said variants comprising at least a minimum percentage sequence identity of at least 85%, at least 90%, at least 93%, at least 96%, at least 97%, at least 98% or at least 99% to the nucleic acid sequence of SEQ ID NO.5, SEQ ID NO. 7, SEQ ID NO. 9 and SEQ ID NO. 11, and wherein said variant(s) encodes a thermostable polypeptide that methylates an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

In some further advantageous embodiments, the DNA-methyltransferase-encoding genes comprise SEQ ID NO.5 and SEQ ID NO. 9 or variants thereof, wherein the nucleic acid sequence of said variants comprising at least a minimum percentage sequence identity of at least 85%, at least 90%, at least 93%, at least 96%, at least 97%, at least 98% or at least 99% to the nucleic acid sequence of SEQ ID NO.5 and SEQ ID NO. 9, and wherein said variant(s) encodes a thermostable polypeptide that methylates an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

The term “gene” refers to a DNA sequence that comprises control and coding sequences necessary for the production of a recoverable bioactive polypeptide or precursor. Endogenous gene are those that originate from within an organism, tissue, or cell.

Furthermore, the present disclosure relates to a method of transforming a microbial cell, the method comprising:
a) treating a DNA molecule comprising at least one 5′-GCATC-3′ sequence with a polypeptide according to the present disclosure or an enzyme composition according to the present disclosure
b) introducing the methylated polynucleotide into a target bacterium.

In particular, the present disclosure pertains to a method for introducing an exogenous DNA molecule into a target bacterium of the species Caldicellulosiruptor saccharolyticus, Caldicellulosiruptor changbaiensis, Caldicellulosiruptor naganoensis and the species or strain Caldicellulosiruptor sp. E32 or of the genus Caldicellulosiruptor sp., wherein a polypeptide having restriction endonuclease activity according to the present disclosure is inhibited by an inhibitor in the bacteria and/or the gene encoding said polypeptide is knocked-out, wherein said inhibitor inhibits the expression of said polypeptide and/or binds to a protein product of a gene coding said polypeptide.

As used herein, the phrase “inhibition of expression” or “inhibits the gene expression” refers to the absence (or observable decrease) in the level of protein and/or mRNA product from the target gene. Specificity refers to the ability to inhibit the target gene without manifest effects on other genes of the cell and without any effects on any gene within the cell that is producing the dsRNA molecule.

The term “Inhibitor” is used as the generic name of the substances inhibiting the expression and/or binds to a protein/polypeptide product of a gene coding the restriction endonuclease according to the present disclosure. For example, the inhibitor may inhibit the expression, the transcription and/or the translation of the restriction endonuclease according to the present disclosure and/or has inhibitory activity against the expressed restriction endonuclease according to the present disclosure.

Therefore, the present disclosure provides recombinant DNA technologies to post-transcriptionally repress or inhibit expression of the restriction endonucleases according to the present disclosure coding sequence in the target bacterium.

A gene knockout is a genetic technique in which one or more of an organism's genes is made inoperative (“knocked out” of the organism), which is presented by Tang et al. (2015).

The following methods and examples are offered for illustrative purposes only, and are not intended to limit the scope of the present disclosure in any way

### METHODS AND EXAMPLES

In the following examples, materials and methods of the present disclosure are provided including the determination of the properties of the microbial strains according to the present disclosure. It should be understood that these examples are for illustrative purpose only and are not to be construed as limiting this disclosure in any manner.

Description of Caldicellulosiruptor sp. Strain BluConL60

Caldicellulosiruptor sp. strain BluConL60 listed in Table 1 was deposited on Aug. 29, 2019 under the accession number DSM 33252 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

Description of Caldicellulosiruptor sp. Strain BluCon006, Caldicellulosiruptor sp. strain BluCon014 and Caldicellulosiruptor sp. Strain BluCon016

Caldicellulosiruptor sp. strain BluCon006, Caldicellulosiruptor sp. strain BluCon014 and Caldicellulosiruptor sp. strain BluCon016, which are listed in Table 2, were deposited on Apr. 9, 2019 under the accession numbers DSM 33095, DSM 33096 and DSM 33097 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

Description of Caldicellulosiruptor sp. Strain DIB104C

Caldicellulosiruptor sp. strain DIB104C listed in Table 2 was deposited on Mar. 15, 2012 under the accession number DSM 25774 according to the requirements of the Budapest Treaty at the Deutsche Sammlung von Mikroorganismen und Zellkulturen (DSMZ), Inhoffenstraße 7B, 38124 Braunschweig (DE) by DIREVO Industrial Biotechnology GmbH, Nattermannallee 1, 50829 Cologne (DE).

Description of Caldicellulosiruptor sp. Strain BluCon085

Caldicellulosiruptor sp. strain BluCon085 was deposited on Mar. 10, 2020 under the accession number DSM 33470 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

Description of Caldicellulosiruptor sp. Strain DIB 104C pyrE M62

Caldicellulosiruptor sp. strain DIB 104C pyrE M62 was deposited on Oct. 22, 2020 under the accession number DSM 33675 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

TABLE 2
Survey of description of Caldicellulosiruptor sp.

| | | | DSMZ | | |
|---|---|---|---|---|---|
| | | | accession | Deposition | |
| Genus | Species | Name | number | date | Depositor |
| Caldicellulosiruptor | sp. | DIB104C | DSM 25774 | Mar. 15, 2012 | DIREVO Industrial |
| | | | | | Biotechnology GmbH |
| | | | | | Nattermannallee 1 |
| | | | | | 50829 Cologne (DE) |
| Caldicellulosiruptor | sp. | BluCon006 | DSM 33095 | Apr. 9, 2019 | BluCon Biotech |
| Caldicellulosiruptor | sp. | BluCon014 | DSM 33096 | Apr. 9, 2019 | GmbH |
| Caldicellulosiruptor | sp. | BluCon016 | DSM 33097 | Apr. 9, 2019 | Nattermannallee 1 |
| Caldicellulosiruptor | sp. | BluConL60 | DSM 33252 | Aug. 29, 2019 | 50829 Cologne (DE) |
| Caldicellulosiruptor | sp. | BluCon085 | DSM 33470 | Mar. 10, 2020 | |
| Caldicellulosiruptor | sp. | DIB 104C | DSM 33675 | Oct. 22, 2020 | |
| | | pyrE M62 | | | |

### Example 1

This example shows that Caldicellulosiruptor sp. DIB 104C and derivatives thereof such as Caldicellulosiruptor sp. BluCon085 have a type II restriction modification (RM) system with an operon structure comprising of one restriction enzyme (encoded by DOCILOBI_02329; SEQ ID NO. 13) and two methyltransferases (encoded by DOCILOBI_02330, SEQ ID NO.7 and DOCILOBI_02331, SEQ ID NO. 11, respectively). This RM-system is homologous to a predicted RM-system of Caldicellulosiruptor saccharolyticus DSM8903, but different from the confirmed RM-system of Caldicellulosiruptor bescii DSM6725 comprising of the restriction enzyme CbeI and the methyltransferase M.CbeI.

### Strains and Cultivation Conditions

Strain Caldicellulosiruptor sp. DIB 104C was deposited on Mar. 15, 2012 under the accession number DSM 25774 according to the requirements of the Budapest Treaty at the Deutsche Sammlung von Mikroorganismen und Zellkulturen (DSMZ), Inhoffenstraße 7B, 38124 Braunschweig (DE) by DIREVO Industrial Biotechnology GmbH, Nattermannallee 1, 50829 Cologne (DE). Strain Caldicellulosiruptor sp. BluCon085 was deposited on Mar. 10, 2020 under the accession number DSM 33470 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

Caldicellulosiruptor cells were routinely cultivated in MOPS-buffered medium containing per liter: NH₄Cl, 1 g; NaCl, 0.5 g; MgSO₄×7H₂O, 0.3 g; CaCl₂×2 H₂O, 0.05 g; NaHCO₃, 0.5 g; KH₂PO₄, 0.1 to 1 g; K₂HPO₄, 0.1 to 1 g; yeast extract, 2 g; MOPS, 1 to 20 g; resazurin, 0.25.10-3 g; vitamin solution, 5 mL; and trace element solution, 1 mL. The vitamin solution contained per liter: biotin, 4 mg; folic acid, 4 mg; pyridoxine hydrochloride, 20 mg; riboflavin, 10 mg; thiamine, 10 mg; nicotinic acid, 10 mg; pantothenic acid, 10 mg; vitamin B12, 0.2 mg; p-aminobenzoic acid, 10 mg; and thioctic acid, 10 mg. The trace element solution contained per liter: HCl (25%; 7.7 M), 10 mL; NiCl₂×6 H₂O, 0.024 g; FeCl₂×4 H₂O, 1.5 g; MnCl₂×4 H₂O, 0.1 g; CoCl₂×6 H₂O, 0.19 g; ZnCl₂, 0.07 g; CuCl₂×2 H₂O, 0.002 g; H₃BO₃, 0.006 g; and Na₂MoO₄·2H₂O, 0.036 g. The carbon source added to the medium was either 5 g/L D-glucose, or 10 g/L Avicel plus 0.5 g/L D-glucose. After dissolving all compounds in water, the pH of the medium was adjusted to 6.4 to 7.4 with 5 M NaOH. The medium was then flushed with N₂ for 20 minutes, after which L-cysteine was added to a final concentration of 0.5 g/L. The medium was subsequently aliquoted in Hungate tubes (Hungate, 1969) or serum bottles that had been flushed with N₂ before. The medium was sterilized by autoclaving at 121° C. and 1 bar overpressure for 20 minutes. Cultures of Caldicellulosiruptor cells were prepared by inoculation of the sterile medium with 0.25 to 1 mL of seed culture. The inoculation was performed under sterile conditions by injection with a syringe through the septum of the Hungate tube or serum bottle. The cultures were subsequently incubated in an orbital shaker at 70° C. and 100 rpm for 24 to 48 hours.

Medium for long-term storage of Caldicellulosiruptor cells contained per liter: D-glucose, 0.5 g; (NH₄)₂SO₄, 1.2 g; MgSO₄×7 H₂O, 0.3 g; CaCl₂×2 H₂O, 0.05 g; NaHCO₃, 0.5 g; K₂HPO₄, 1.5 g; KH₂PO₄, 3 g; MOPS, 1 to 40 g; yeast extract, 2 g; resazurin, 0.25.10-3 g; vitamin solution, 5 mL; and trace element solution, 1 mL. After dissolving all compounds in water, the pH of the medium was adjusted to 6.4 to 7.4 with 5 M NaOH. The medium was then flushed with N₂ for 20 minutes, after which L-cysteine was added to a final concentration of 1 g/L. In total 9 mL of the medium were transferred into Hungate tubes containing a strip of Whatman #1 filter paper (1 to 6 cm corresponding to approximately 50 mg). The tubes with filter paper had been flushed with N₂ before and incubated at room temperature for approximately one hour to remove the oxygen from the filter paper. After transferring the medium into the tubes, the medium was sterilized by autoclaving at 121° C. and 1 bar overpressure for 20 minutes. Cultures of Caldicellulosiruptor cells were prepared by inoculation of the sterile medium with 0.25 to 0.5 mL of seed culture. The cultures were subsequently incubated in a static incubator at 70° C. until initial decomposition of the filter paper was observed (usually after one to two days). The tubes were stored at −20 to −28° C.

Restriction Endonuclease Digestion of Caldicellulosiruptor sp. DIB 104C Genomic DNA Shows a Different Pattern as Compared to Caldicellulosiruptor bescii DSM6725 Genomic DNA

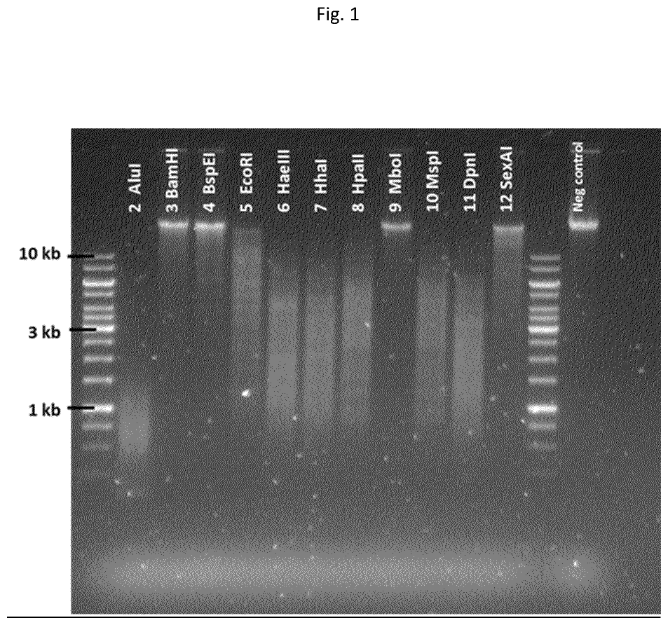

Genomic DNA from Caldicellulosiruptor sp. DIB 104C was isolated using the MasterPure Gram Positive DNA Purification Kit from Epicentre according to the manufacturer's instructions. The genomic DNA was subsequently digested using the methylation-sensitive restriction enzymes AluI, BamHI, BspEI, EcoRI, HaeIII, HhaI, HpaII, MboI, MspI, DpnI and SexAI. In particular, 1 g of genomic DNA in 20 μl was incubated with 10 units of each restriction enzyme (New England BioLabs) in the appropriate buffer for 2 hours. The same reaction without restriction enzyme served as the negative control. The digestion products were then separated on a 0.7% agarose gel by electrophoresis and stained with SYBR Safe DNA Gel Stain (Invitrogen). The results show that genomic DNA from Caldicellulosiruptor sp. DIB 104C is digested by the restriction enzymes AluI, EcoRI, HaeIII, HhaI, HpaII, MspI and DpnI, but not by the restriction enzymes BamHI, BspEI, MboI and SexAI (FIG. 1 and Table 1).

TABLE 1
Digestion of genomic DNA from
Caldicellulosiruptor sp. DIB
104C with methylation-sensitive
restriction enzymes. For each
enzyme, the recognition site is
given. The “+” sign means that
the genomic DNA was digested;
the “−” sign means that the
genomic DNA was not digested.

| Restriction | Recognition | |
|---|---|---|
| enzyme | site | Digestion |
| AluI | 5′-AGCT-3′ | + |
| BamHI | 5′-GGATCC-3′ | − |
| BspEI | 5′-TCCGGA-3′ | − |
| EcoRI | 5′-GAATTC-3′ | + |
| HaeIII | 5′-GGCC-3′ | + |
| HhaI | 5′-GCGC-3′ | + |
| HpaIl | 5′-CCGG-3′ | + |
| MboI | 5′-GATC-3′ | − |
| MspI | 5′-CCGG-3′ | + |
| DpnI | 5′-GATC-3′ | + |
| SexAI | 5′-ACCWGGT-3′ | − |

In a next step, the restriction endonuclease digestion pattern of strain Caldicellulosiruptor sp. DIB 104C was compared to that of seven other Caldicellulosiruptor strains previously published by Chung et al. (2013) (Table 2). We highlight here the result for the restriction enzyme HaeIII that digests genomic DNA from C. obsidiansis ATCC BAA-2073, C. lactoaceticus DSM9545, C. kronotskyensis DSM12137 and C. sp. DIB 104C, but not from C. bescii DSM6725, C. hydrothermalis DSM18901, C. kristijansonii DSM12137 and C. saccharolyticus DSM8903.

The relevance of the HaeIII digestion pattern relates to the fact that C. bescii DSM6725 has a potent restriction endonuclease, referred to as CbeI, which is an isoschizomer of HaeIII that cleaves unmethylated sequences at 5′-GGCC-3′ (Chung et al., 2011). CbeI has been shown to be a barrier to DNA transformation of C. bescii, and deletion of the gene encoding this restriction enzyme has been shown to lead to successful transformation without the need for prior methylation of the template DNA (Chung et al., 2013). As genomic DNA from C. bescii DSM6725 is not digested by HaeIII, the respective sequence is expected to be methylated in this strain. The fact that HaeIII digests genomic DNA from Caldicellulosiruptor sp. DIB 104C suggests that a different RM-system is active in this strain as compared to C. bescii DSM6725.

TABLE 2
Digestion of genomic DNA from different Caldicellulosiruptor strains with methylation-sensitive
restriction enzymes. The information for the first 7 strains was taken from Chung et al. (2013).

| | Restriction enzyme | | | | | | | | | | |
|---|---|---|---|---|---|---|---|---|---|---|---|
| Strain | AluI | BamHI | BspEI | EcoRI | HaeIII | HhaI | HpaII | MboI | MspI | DpnI | SexAI |
| C. bescii | + | − | − | + | − | + | + | + | + | n.d. | n.d. |
| DSM6725 | | | | | | | | | | | |
| C. hydrothermalis | + | − | − | + | − | + | + | + | + | n.d. | n.d |
| DSM18901 | | | | | | | | | | | |
| C. kristijansonii | + | − | − | + | − | + | + | − | + | n.d. | n.d. |
| DSM12137 | | | | | | | | | | | |
| C. saccharolyticus | + | − | − | + | − | + | + | − | + | n.d. | n.d. |
| DSM8903 | | | | | | | | | | | |
| C. obsidiansis | + | − | − | + | + | + | + | − | + | n.d. | n.d. |
| ATCC BAA-2073 | | | | | | | | | | | |
| C. lactoaceticus | + | − | − | + | + | + | + | − | + | n.d. | n.d |
| DSM9545 | | | | | | | | | | | |
| C. kronotskyensis | + | − | − | + | + | + | − | − | − | n.d. | n.d. |
| DSM12137 | | | | | | | | | | | |
| C. sp. DIB 104C | + | − | − | + | + | + | + | − | + | + | − |

The “+” sign means that the genomic DNA was digested; the “−” sign means that the genomic DNA was not digested.

Genome-Wide Analysis to Identify a Type II RM-System in Caldicellulosiruptor sp. BluCon085
a. Absence of Homologous Genes Encoding the CbeI/M.CbeI RM-System of Caldicellulosiruptor bescii DSM6725 in the Genome of Caldicellulosiruptor sp. BluCon085

The methyltransferase M.CbeI of Caldicellulosiruptor bescii DSM6725 has been described by Chung et al. (2012) as an “α-class N4-cytosine methyltransferase”. According to the REBASE database with information on restriction enzymes and methyltransferases (http://rebase.neb.com/rebase/rebase.html; Roberts et al., 2010), α-class type II methyltransferases contain the conserved “fgg-TRD-dppy” sequence (http://rebase.neb.com/cgi-bin/sublist).

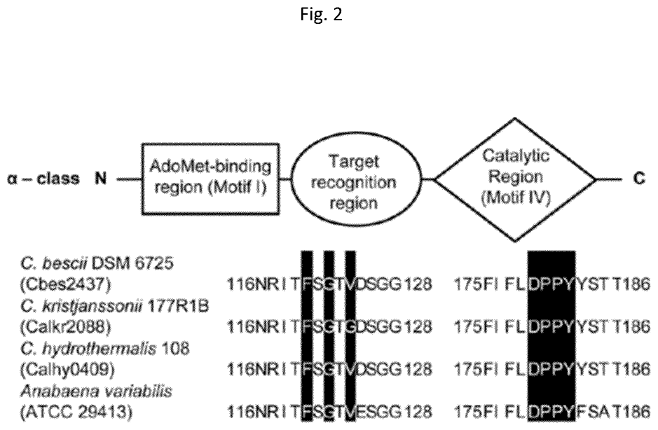

An alignment of M.CbeI homologous methyltransferases from different genera and species has identified a target recognition domain (TRD) with the motif “FSGTV” and a catalytic domain with the motif “DPPY” (Chung et al., 2012) (FIG. 2). The motif “DPPY” is common for almost all methyltransferases. Therefore, we anticipate that the identification of genes encoding for M.CbeI homologous methyltransferases in the genome of Caldicellulosiruptor sp. BluCon085 should focus on the identification of the TRD with the motif “FSGTV”.

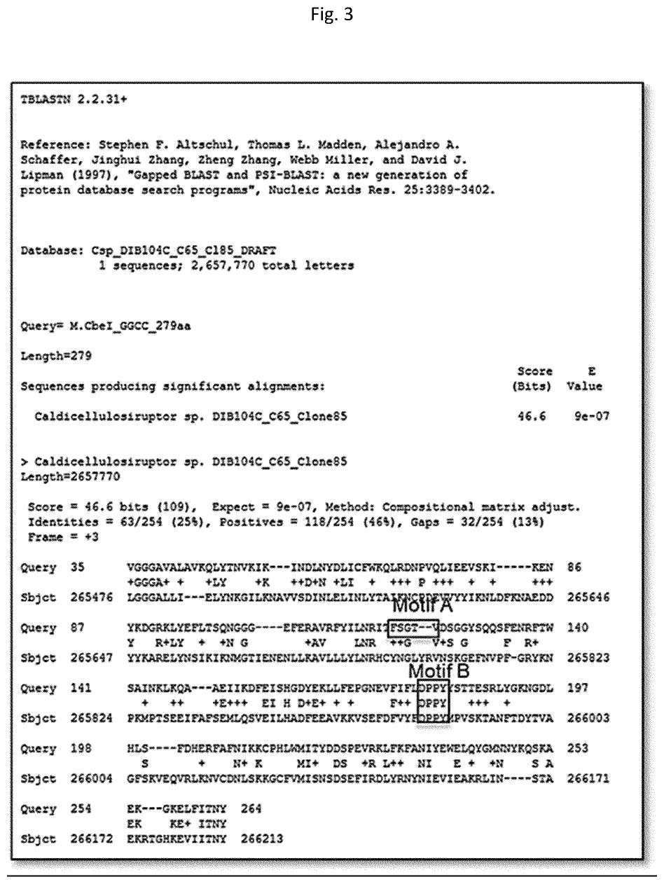

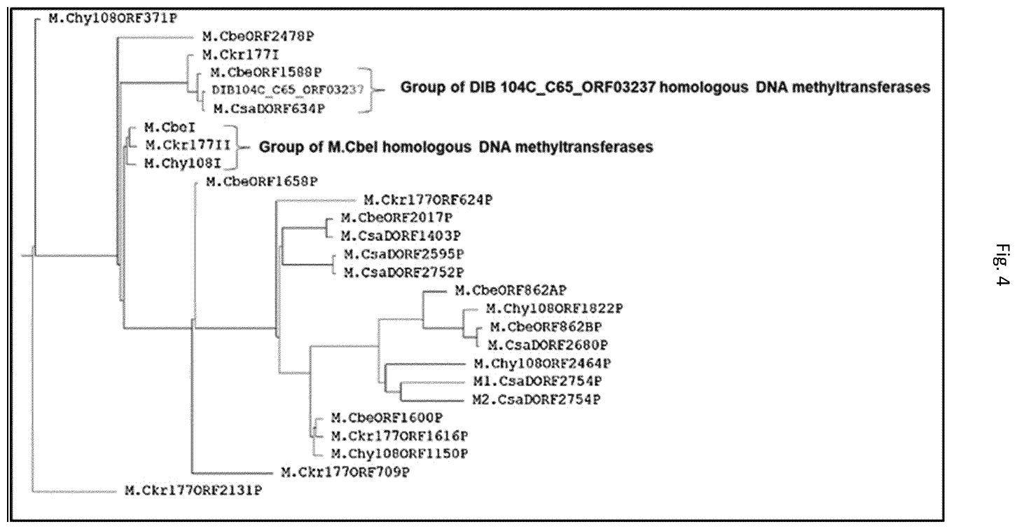

To identify a M.CbeI homologous methyltransferase in Caldicellulosiruptor sp. BluCon085, we first extracted the genomic DNA of the strain using the MasterPure Gram Positive DNA Purification Kit (Epicentre), and then determined the whole-genome sequence of the strain by Nanopore and Illumina sequencing. Next, a tblastn analysis was carried out (https://blast.ncbi.nlm.nih.gov/Blast.cgi?PROGRAM=tblastn&PAGE_TYPE=BlastSearch&LINL_LOC=blasthome), in which the genome sequence of Caldicellulosiruptor sp. BluCon085 was translated into 6 reading frames (both strands) and the generated proteome compared to the protein sequence of the methyltransferase M.CbeI. The protein identified as the best hit (FDFOHOFN_03237 (ORF03237, DRAFT Genome)) contained only the catalytic domain with the motif “DPPY”; the TRD with the motif “FSGTV” was not present (FIG. 3).

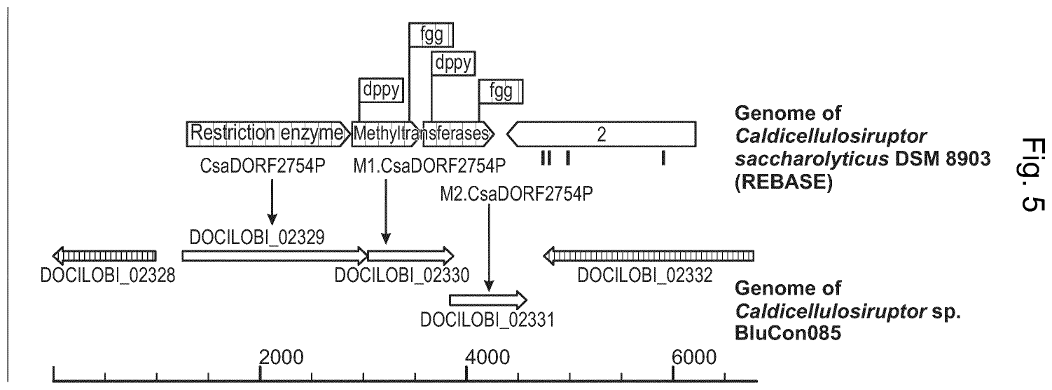

The protein sequence of the best-hit protein was compared in a multiple alignment with all methyltransferases from the genus Caldicellulosiruptor as annotated in REBASE. In the family tree representation (program Clone Manager from Sci-Ed Software), the best-hit protein did not cluster in the group of M.CbeI homologous methyltransferases (FIG. 4). This result reinforces our assumption that Caldicellulosiruptor sp. BluCon085 does not encode for a methyltransferase homologous to that of the CbeI/M.CbeI RM-system, and that a different RM-system is active in this strain.

b. Identification of a Type IIS RM-System in Caldicellulosiruptor sp. BluCon085

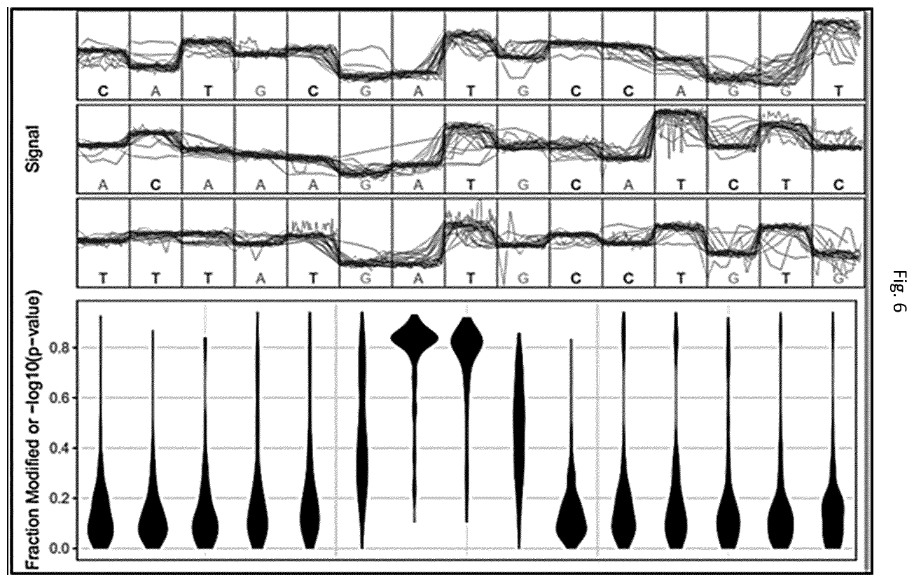

A phylogenetic tree of Caldicellulosiruptor strains published by Svetlitchnyi et al. (2013) shows that Caldicellulosiruptor sp. DIB 104C is most closely related to the type strain Caldicellulosiruptor saccharolyticus DSM8903. For the latter strain, a type II RM-system operon structure is predicted in REBASE that consists of the restriction enzyme CsaDORF2754P and the methyltransferases M1.CsaDORF2754P and M2.CsaDORF2754P (FIG. 5). The recognition sequence of the methyltransferases is predicted to be “GANTC”. A blastn analysis of the three respective genes against the genome sequence of Caldicellulosiruptor sp. BluCon085 has identified genes encoding a homologous type II RM-system in this strain: DOCILOBI_02330 and DOCILOBI_02331 encode for methyltransferases, and DOCILOBI_02329 for an endonuclease (FIG. 5).

With regard to the gene DOCILOBI_02329 encoding for an endonuclease, an automated annotation of the Caldicellulosiruptor sp. BluCon085 genome sequence has pinpointed this endonuclease as an “AlwI family type II restriction endonuclease”. Members of this family generally recognize asymmetric methylated sequences including GGATC (AlwI), GCNGC (Bsp6I), GASTC (BsTNBI) and GAGTC (PleI and MlyI) (Pingoud et al., 2014). The AlwI RM-system consists of one endonuclease and two methyltransferases joined into a single chain—one specific for the top-strand sequence, the other for the complementary bottom-strand sequence.

The information on the homology with the type II RM-system of C. saccharolyticus DSM8903 and the annotation of the endonuclease as a AlwI family type II restriction endonuclease suggests that the RM-system in Caldicellulosiruptor sp. BluCon085 is a type IIS system that consists of one endonuclease and two strand-specific methyltransferases that recognize asymmetric methylated sequences.

### Example 2

This example shows that the predicted recognition sites of the RM-system in Caldicellulosiruptor sp. BluCon085 and variants thereof such as Caldicellulosiruptor sp. DIB 104C are 5′-GCATC-3′ and its complementary sequence 5′-GATGC-3′. We show that the methyltransferases of the RM-system methylate the adenine in the two recognition sites (m6A), resulting in an asymmetric methylation of the genomic DNA.

### Identification of Base Modifications Using Nanopore Sequencing: Sequence Motifs 5′-GATGC-3′ and 5′-GCATC-3′

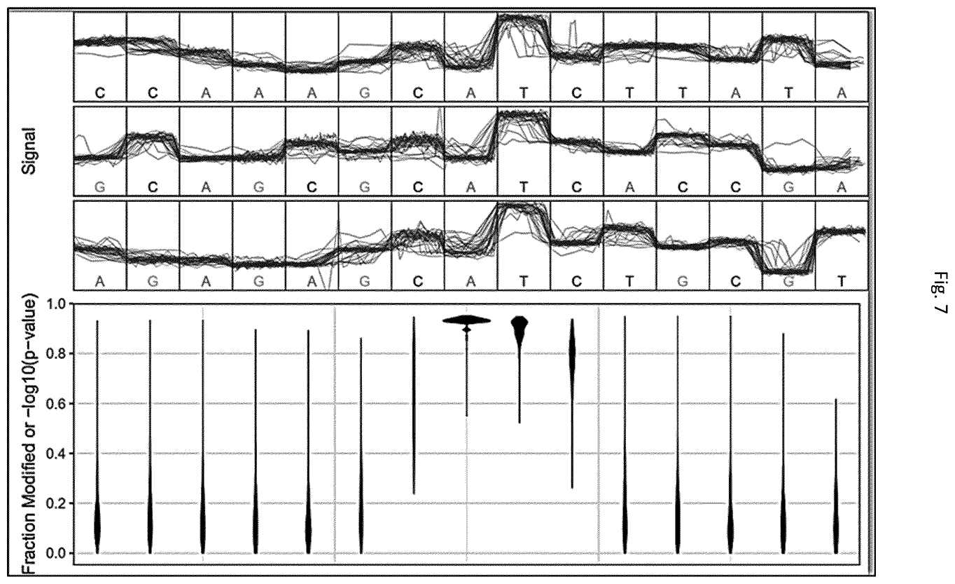

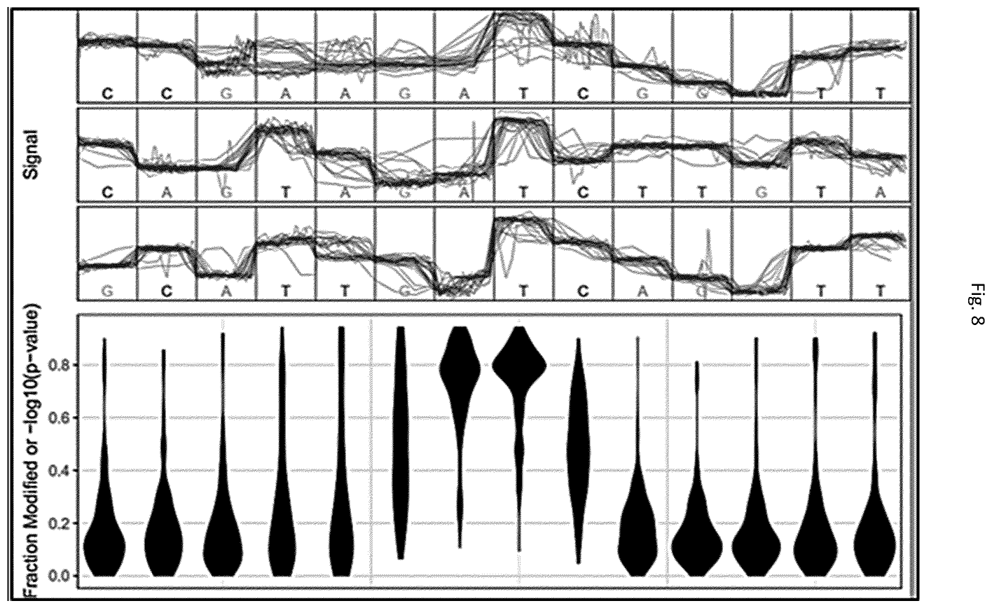

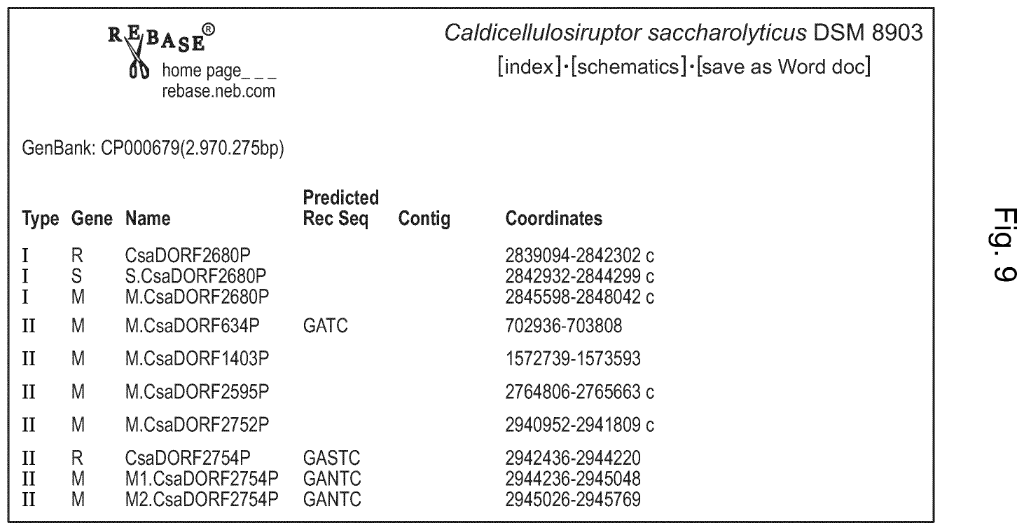

The Nanopore dataset from strain Caldicellulosiruptor sp. BluCon085 (see Example 1) was analysed using the “Tombo” software to identify the methylated positions in the genomic DNA. In particular, the “Tombo” software compares and evaluates the base-specific voltage change of the analysed single-stranded DNA on the pores of the Nanopore device, in this way allowing the de novo identification of modified bases such as 5-methylcytosine (m5C) and N6-methyladenosine (m6A) methylations. The graphical results relevant for this example are presented in the violin plots in FIGS. 6 and 7. Within the sequence motifs 5′-GATGC-3′ (FIG. 6) and 5′-GCATC-3′ (FIG. 7), the motif “AT” is methylated, in particular the base adenine is methylated in the sequence motifs 5′-GATGC-3′ and 5′-GCATC-3′.

### Additional Base Modification Identified Using Nanopore Sequencing: Sequence Motif 5′-GATC-3′

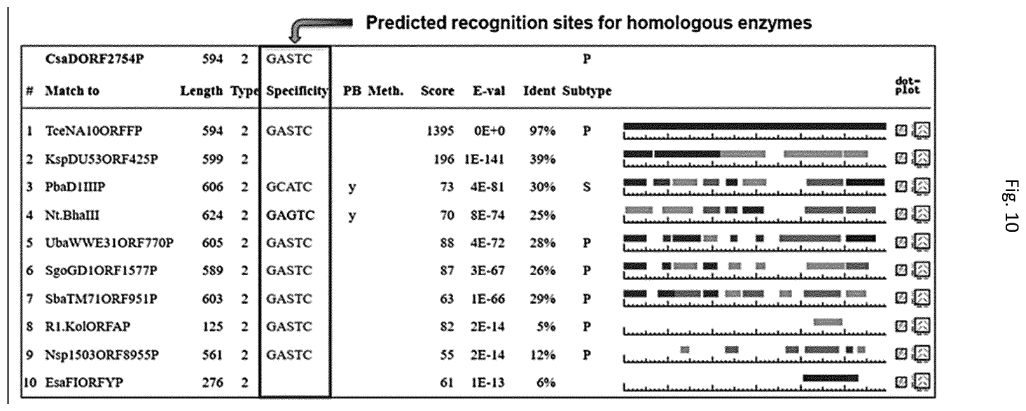

In addition to the sequence motifs 5′-GATGC-3′ and 5′-GCATC-3′, analysis of the Nanopore dataset using the “Tombo” software also identified the sequence motif 5′-GATC-3′ as a probable methylated motif in the genome of Caldicellulosiruptor sp. BluCon085. The graphical result is presented in the violin plot in FIG. 8. For the same reason as given for the sequence motifs 5′-GATGC-3′ and 5′-GCATC-3′, the base adenine is most likely methylated in the sequence motif 5′-GATC-3′. A total of 2095 methylated 5′-GATC-3′ motifs were detected in the genome of Caldicellulosiruptor sp. BluCon085.

The presence of a methylated adenine of the sequence motif 5′-GATC-3′ was verified by digestion of the genomic DNA from Caldicellulosiruptor sp. DIB 104C with the methylation-sensitive restriction enzymes DpnI and MboI (see Example 1). DpnI cuts the sequence motif 5′-GATC-3′ only when m6A methylation is present, whereas MboI does not cut the sequence motif when m6A methylation is present. Our results show that the base adenine in the sequence motif 5′-GATC-3′ is indeed methylated in Caldicellulosiruptor sp. DIB 104C (Example 1, FIG. 1 (Lanes 9 and 11) and Table 1).

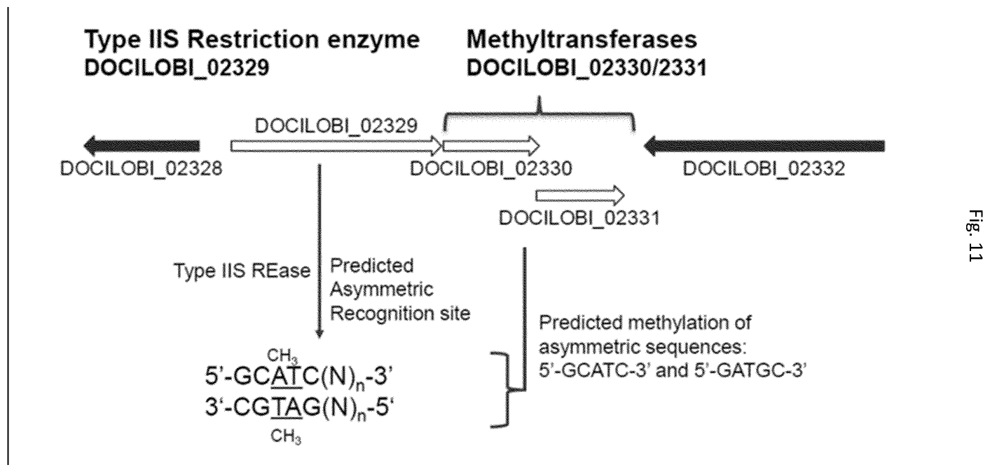

Although the motif 5′-GATC-3′ is methylated in Caldicellulosiruptor sp. DIB 104C and derivatives thereof, it most probably does not represent a functional RM-system in this strain. The main reason is that the methyltransferase expected to methylate the motif 5′-GATC-3′ is not located in close proximity to a restriction enzyme, as is the case for most potent RM-systems in Bacteria (Wilson et. al., 2012). In fact, the methyltransferase with the predicted recognition sequence 5′-GATC-3′ in Caldicellulosiruptor saccharolyticus DSM 8903 is M.CsaDORF634P, which, according to the REBASE database, is not paired with a restriction enzyme (FIG. 9). The homologous gene in Caldicellulosiruptor sp. BluCon085 (DOCILOBI_01978) is also not paired with a restriction enzyme.

In bacteria such as in Escherichia coli K-12 and other Gammaproteobacteria, the methylation of adenine at the N6 position (m6A) of the GATC consensus sequence is catalysed by a DNA adenine methyltransferase (Dam). Methylation of this adenine modulates cellular processes including transcriptional regulation of gene expression, initiation of chromosomal replication, and DNA mismatch repair (Westphal et al., 2016). These cellular processes may be modulated in a similar manner in Caldicellulosiruptor sp. DIB 104C and derivatives thereof.

Prediction of the Recognition Site of the Type IIS RM-System in Caldicellulosiruptor sp. DIB 104C

A blastp analysis to identify restriction enzymes homologous to the restriction enzyme encoded by CsaDORF2754P from Caldicellulosiruptor saccharolyticus DSM8903 was carried out in REBASE in order to predict the recognition site of the enzyme (FIG. 10). Among the identified restriction enzymes, PbaD1IIIP recognizes the sequence motif 5′-GCATC-3′; the corresponding methyltransferase is known to modify the position adenine (m6A). This sequence motif is the same as that identified by the violin plots (FIG. 7), suggesting that the recognition and methylation sites of the type IIS RM-system of Caldicellulosiruptor sp. BluCon085 are 5′-GCATC-3′ and its complementary sequence 5′-GATGC-3′, both containing a m6A methylation (FIG. 11).

Verification of the Predicted Recognition and Methylation Site 5′-GCATC-3′ of the Annotated Type IIS RM-System in Caldicellulosiruptor sp. BluCon085

To verify the predicted recognition and methylation sites, methylated genomic DNA from Caldicellulosiruptor sp. BluCon085 and unmethylated plasmid DNA pF25-5br (SEQ ID NO: 1) isolated from Escherichia coli INV110 (a dam- and dcm-deficient strain) were digested with various adenosine methylation-sensitive and adenosine methylation-insensitive restriction enzymes. The methylation-sensitive enzymes and their recognition sites are: AlwI (site 5′-GGATC(N)₄-3′) (Nelson et al., 1993), SfaNI (site 5′-GCATC(N)₅-3′) (Nelson et al., 1993) and DpnI (site 5′-GATC-3′); the methylation-insensitive enzyme and its recognition site is: PleI (site 5′-GAGTC(N)₄-3′).

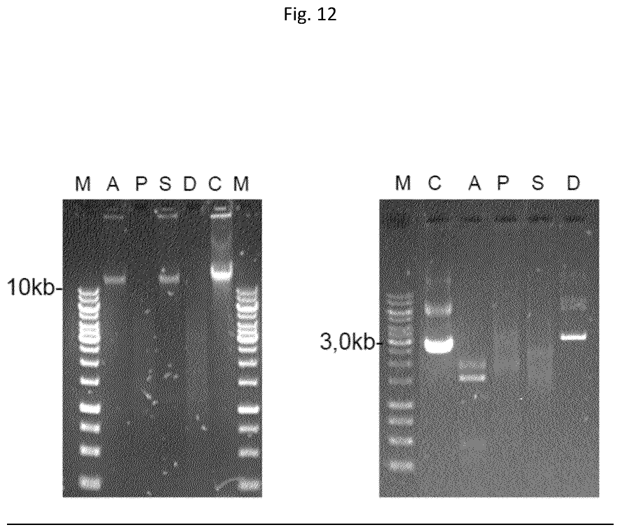

In total 2 μg of DNA in 50 μl were incubated with 10 units of each restriction enzyme (New England BioLabs) in the appropriate buffer for 2 hours. The same reaction without restriction enzyme served as the negative control. The reaction products were then separated on a 0.7% agarose gel by electrophoresis, after which the DNA was stained with SYBR Safe DNA Gel Stain (Invitrogen). Our results show that the methylated genomic DNA from Caldicellulosiruptor sp. BluCon085 is hydrolysed by the restriction enzymes PleI and DpnI, but not by the restriction enzymes AlwI and SfaNI (FIG. 12 and Table 3). The unmethylated plasmid DNA pF25-5br is hydrolysed by the restriction enzymes PleI, AlwI and SfaNI, but not by the restriction enzyme DpnI (FIG. 12 and Table 3).

The restriction enzyme DpnI only recognizes and cleaves the motif 5′-GATC-3′ when the adenine in the motif is methylated (m6A methylation). The fact that genomic DNA from Caldicellulosiruptor sp. BluCon085 is hydrolysed by DpnI thus shows that the respective motif is indeed methylated in this strain, as suggested before by the violin plot in FIG. 10. This conclusion is further supported by the fact that genomic DNA from Caldicellulosiruptor sp. BluCon085 is not cleaved by AlwI. In fact, recognition and cleavage by AlwI is prevented by m6A methylation of the motif sequence 5′-GGATC(N)₄-3′, which includes the motif sequence 5′-GATC-3′.

SfaNI digestion is prevented by m6A methylation of the motif sequence 5′-GCATC(N)₅-3′. The fact that genomic DNA from Caldicellulosiruptor sp. BluCon085 is not hydrolysed by SfaNI thus shows that the motif sequence is indeed methylated in this strain. Therefore, the motif of the recognition site for the RM-system of Caldicellulosiruptor sp. BluCon085 is 5′-GCATC-3′ with m6A methylation and the complementary sequence of 5′-GCATC-3′, which is 5′-GATGC-3′, in which the adenine is methylated as well. Methylation of the adenine bases in both sequences is concomitant with the asymmetric methylation of the genomic DNA of Caldicellulosiruptor sp. BluCon085.

Because strain Caldicellulosiruptor sp. DIB 104C is the parent strain of strain Caldicellulosiruptor sp. BluCon085, the RM-system in the former strain is expected to be the same as in the latter strain. This implies, by extension, for all strains that are derived from Caldicellulosiruptor sp. DIB 104C and BluCon085.

TABLE 3
Digestion of genomic DNA from Caldicellulosiruptor sp. BluCon085
and plasmid DNA pF25-5br with methylation-sensitive restriction
enzymes. For each enzyme, the recognition site is given.

| | | Digestion | |
|---|---|---|---|
| | | Methylated DNA | Unmethylated DNA |
| Restriction enzyme | Recognition site | C. sp. BluCon085 | plasmid DNA pF25-5br |
| AlwI | 5′-GGATC(N)₄-3′ | − | + |
| PleI | 5′-GAGTC(N)₄-3′ | + | + |
| SfaNI | 5′-GCATC(N)₅-3′ | − | + |
| DpnI | 5′-GATC-3′ | + | − |
| Control (without enzyme) | -.- | − | − |

The “+” sign means that the genomic DNA was digested; the “−” sign means that the genomic DNA was not digested.

### Example 3

This example shows that in vitro methylation of a DNA fragment using the methyltransferases EcMt1ORF02330 (SEQ ID NO: 6) and EcMt2ORF02331 (SEQ ID NO: 10) and S-adenosyl-methionine as a source of methyl groups prevents restriction by a cell lysate of Caldicellulosiruptor sp. BluCon085, a derivative of Caldicellulosiruptor sp. DIB 104C.

### Strains and Cultivation Conditions

A description of the strains Caldicellulosiruptor sp. DIB 104C and Caldicellulosiruptor sp. BluCon085 is presented in Example 1. The strains Escherichia coli NEB 10-beta pTrc-EcMt1ORF02330 and pTrc-EcMt2ORF02331 were obtained by transformation of the strain NEB 10-beta (New England BioLabs) with the plasmids pTrc-EcMt1ORF02330 (containing the ORF from gene DOCILOBI_02330; SEQ ID NO: 6) and pTrc-EcMt2ORF02331 (containing the ORF from gene DOCILOBI_02331; SEQ ID NO: 3), respectively, applying electroporation according to Sambrook and Russel (2001).

Escherichia coli cells were routinely cultivated in LB medium containing per liter: tryptone, 10 g; yeast extract, 5 g; and NaCl, 10 g. For solid medium, 30 g of agar were added. After dissolving all compounds in water, the medium was sterilized by autoclaving at 121° C. and 1 bar overpressure for 20 minutes. Cultures of Escherichia coli cells were prepared by inoculation of the sterile medium with cells from a single cell colony picked from plate. The cultures were subsequently incubated in an orbital shaker at 37° C. and 200 rpm.

Preparation of a Cell Lysate from Caldicellulosiruptor sp. BluCon085

A cell lysate from Caldicellulosiruptor sp. BluCon085 was prepared from cells grown to mid-logarithmic phase in 500 mL of MOPS-buffered medium. The cells were harvested by centrifugation for 15 minutes at 6,000×g and 4° C., and resuspended in 500 l of CelLytic B Cell Lysis Reagent (Sigma-Aldrich) containing a protease inhibitor cocktail (cOmplete from Roche). The cell suspension was subsequently sonicated on ice using a Branson Sonifier 250 with output control of 2 and duty cycle of 50%. The lysis process encompassed three cycles of each 10 seconds of sonification followed by 10 seconds of cooling on ice. The cell lysate was then centrifuged for 15 minutes at 13,000 rpm and 4° C. Supernatants were used immediately for enzyme activity assays.

### Expression of Methyltransferases EcMt1ORF02330 and EcMt2ORF02331

Cells from a single cell colony of E. coli NEB 10-beta strains harbouring expression vectors pTrc-EcMt1ORF02330 and pTrc-EcMt2ORF02331, respectively, were used to inoculate 5 mL of LB medium containing 100 μg/mL ampicillin. The cells were subsequently incubated overnight in an orbital shaker at 250 rpm and 37° C. An appropriate amount of cell culture to obtain an optical density at 600 nm (OD_{600 nm}) of 0.1 was transferred into 50 mL of LB medium containing ampicillin, and incubated in an orbital shaker at 250 rpm and 37° C. until an OD_{600 nm} of 0.4 was reached. Then, expression of the methyltransferases EcMt1ORF02330 and EcMt2ORF02331 was induced by addition of 0.5 mM IPTG and incubation in an orbital shaker at 250 rpm and 28° C. The next day, an appropriate amount of cell culture to obtain an OD_{600 nm} of 50 in 1 mL was transferred into a 50-mL tube, and cells were collected by centrifugation for 10 minutes at 4,000×g and 10° C.

Extraction and Heat Purification of Methyltransferases from E. coli Cell Lysates

The cell pellets obtained after expression of the methyltransferases were resuspended in 1 mL of ice-cold lysis buffer. The lysis buffer contained per 40 mL: 1 tablet of cOmplete EDTA-free protease inhibitor cocktail (Roche), 4 mL of 0.5 M EDTA, 0.04 mL of 1 M DTT and 4 mL of 10× CutSmart buffer (New England BioLabs). The cells were subsequently lysed using a Branson Sonifier 250. After the lysis process, the cell debris was removed by centrifugation for 5 minutes at 10,000×g and room temperature. The supernatant was transferred into a 1.5-mL reaction tube, and subsequently incubated for 10 minutes at 70° C. in order to purify the methyltransferases EcMtORF02330 and EcMt2ORF02331 from the E. coli cell lysates. The cell debris was removed once again by centrifugation for 5 minutes at 10,000×g.

### In Vitro Methylation of a Linear DNA Fragment

In a first step, a linear DNA fragment containing the pyrE gene was constructed by PCR. In particular, the primers MCUP180 (sequence 5′-AGATCAAAGGATCTTCTTGAGATC-3′) and BLU32 (sequence 5′-AAGAAATAGCGGTCTGACGCTCAGTGGAACG-3′) were used to amplify the pyrE gene from plasmid pF25-6brBglII (SEQ ID NO: 4). The PCR reaction was performed using PhusionFlash Mastermix (ThermoFisher) and the PCR product was purified from the reaction mixture using the DNA Clean and Concentrate Kit (Zymo Research).

The DNA fragment obtained by PCR was subsequently methylated using the purified methyltransferases EcMtORF02330 and EcMt2ORF02331 and S-adenosyl-methionine (32 mM) as a source of methyl groups.

### Purification of Methylated DNA by Phenol/Chloroform Extraction and Isopropanol Precipitation

The volume of the methylation reaction was adjusted to 300 μL using ultrapure water. Then, 300 μL of phenol:chloroform:isoamylalcohol (PCI; 25:24:1) was added and mixed with the methylation reaction mixture by thoroughly inverting the tube for 1 minute. After 10 minutes of centrifugation at >10,000×g and room temperature, 250 μL of the upper layer containing the DNA were transferred into a new 2-mL reaction tube and 250 μL of PCI was added. After mixing for 1 minute, the mixture was once again centrifuged at >10,000×g and room temperature. In total 200 μL of the upper layer were transferred into a 1.5-mL reaction tube. Next, 200 μL of isopropanol and 20 μL of NaOAc (3 M) were added to the DNA mixture, and the solution was mixed by inverting the tube. After 30 minutes of incubation at room temperature, the DNA was collected by centrifugation for 15 minutes at >10,000×g and room temperature. Then, the DNA pellet was washed with 200 mL of 70% ethanol, and subsequently dried in a heat block at 40° C. The DNA pellet was resuspended in 50 L of ultrapure water.

Methylation Using the Methyltransferases EcMt1ORF02330 and EcMt2ORF02331 Protects DNA from Endogenous Nucleases Present in a Cell Lysate from Caldicellulosiruptor sp. BluCon085

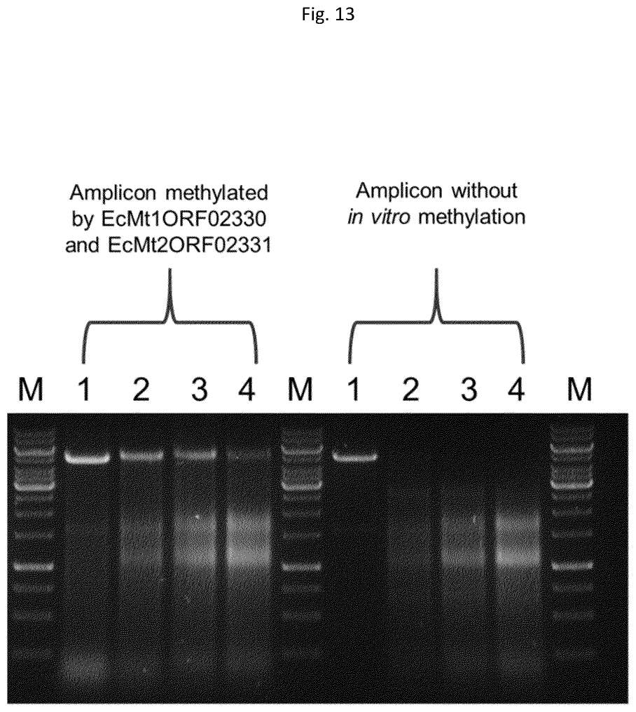

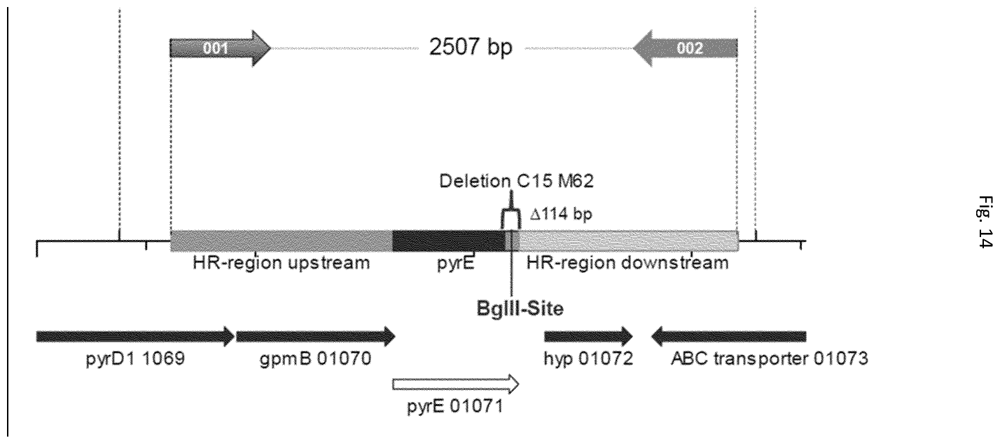

The methylated DNA fragment was incubated in the presence of a Caldicellulosiruptor sp. BluCon085 cell lysate to assess degradation of the fragment by endogenous nucleases. The reactions were performed in a final volume of 14 μl containing 0.5 μg of DNA and either 14% (2 μl), 29% (4 μl) or 43% (6 μl) (v/v) of cell lysate. Adjustment of the volume to the final volume was performed with CutSmart buffer (New England BioLabs) containing 1 mM DTT. The reaction mixtures were subsequently incubated at 70° C. for 30 minutes. As a reference, the unmethylated DNA fragment was treated under the same conditions. After incubation, the reaction products were separated on a 1% agarose gel by electrophoresis and DNA was stained with SYBR Safe DNA Gel Stain (Invitrogen) (FIG. 13). The size of the full-length DNA fragment is 4956 bps.

Our results show that methylation of a linear DNA fragment using the methyltransferases EcMt1ORF02330 and EcMt2ORF02331 partly protects the fragment from restriction by endogenous nucleases present in a Caldicellulosiruptor sp. BluCon085 cell lysate. A linear DNA fragment that is not methylated using the methyltransferases was degraded within the 30-minutes time frame of the experiment.

### Example 4

This example describes a transformation experiment in which a pyrE gene repair fragment was introduced into the Caldicellulosiruptor sp. DIB 104C pyrE M62 strain, a derivative of Caldicellulosiruptor sp. DIB 104C that contains a partial deletion of the pyrE gene making the gene non-functional. The pyrE repair fragment was provided either on a supercoiled plasmid or as a linear PTO-protected PCR-amplified fragment. Both DNA molecules showed a compatible methylation pattern obtained by in vitro methylation using the methyltransferases EcMt1ORF02330 (SEQ ID NO: 6) and EcMt2ORF02331 (SEQ ID NO: 10) and S-adenosyl-methionine as a source of methyl groups. A BglII recognition site was introduced immediately downstream of the pyrE open reading frame for easy detection of successful repair events.

### Strains and Cultivation Conditions

Caldicellulosiruptor sp. DIB 104C pyrE M62 was deposited on Oct. 22, 2020 under the accession number DSM 33675 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

The LOD medium used for the generation of competent cells and the recovery of cells after transformation was created according to Farkas et al., 2013. For solid medium, 30 g of agar were added. After dissolving all compounds in water. The medium was flushed with N₂ for 20 minutes, after which L-cysteine was added to a final concentration of 1 g/L. The medium was subsequently aliquoted in Hungate tubes (Hungate, 1969) or serum bottles that had been flushed with N₂ before. The medium was sterilized by autoclaving at 121° C. and 1 bar overpressure for 20 minutes. Cultures of Caldicellulosiruptor cells were prepared by inoculation of the sterile medium with a seed culture. The inoculation was performed under sterile conditions by injection with a syringe through the septum of the Hungate tube or serum bottle. The cultures were subsequently incubated in an orbital shaker at 70° C. and 100 rpm.

The MOPS-buffered medium with filter paper and CSM-Ura used for the enrichment of uracil prototrophic transformants contained the following per liter: D-glucose, 0.5 g; (NH₄)₂SO₄, 1.2 g; MgSO₄×7 H₂O, 0.3 g; CaCl₂×2 H₂O, 0.05 g; NaHCO₃, 0.5 g; K₂HPO₄, 0.1 to 1 g; KH₂PO₄, 0.1 to 1 g; CSM-Ura, 0.77 g; MOPS, 1 to 20 g; resazurin, 0.25.10-3 g; vitamin solution, 5 mL; and trace element solution, 1 mL. After dissolving all compounds in water, the pH of the medium was adjusted to 6.4 to 7.4 with 5 M NaOH. The medium was then flushed with N₂ for 20 minutes, after which L-cysteine was added to a final concentration of 1 g/L. In total 9 mL of the medium were transferred into Hungate tubes containing a strip of Whatman #1 filter paper (1 to 6 cm corresponding to approximately 50 mg). The tubes with filter paper had been flushed with N₂ before and incubated at room temperature for approximately one hour to remove the oxygen from the filter paper. After transferring the medium into the tubes, the medium was sterilized by autoclaving at 121° C. and 1 bar overpressure for 20 minutes. Cultures of Caldicellulosiruptor cells were prepared by inoculation of the sterile medium with a seed culture. The cultures were subsequently incubated at 70° C.

The MOPS-buffered medium with filter paper and yeast extract has the same composition as the MOPS-buffered medium with filter paper and CSM-Ura, with the exception that 5 g/L of yeast extract was used instead of CSM-Ura.

### In Vitro Methylation of Supercoiled Plasmid DNA and PTO-Protected Linear DNA

Two different DNA templates were prepared for methylation and subsequent transfer into Caldicellulosiruptor sp. DIB 104C pyrE M62. The first template concerns the plasmid pF25-6brBglII (SEQ ID NO: 4) containing the pyrE open reading frame and 1005-bp upstream and 926-bp downstream regions. Immediately downstream of the open reading frame, a cytosine was inserted into the DNA thus creating a BglII recognition site that can be used as an efficient marker for a successful integration event (FIG. 14). The plasmid pF25-6brBglII was isolated from the respective E. coli strain using the QIAprep Spin Miniprep kit (Qiagen). The second template concerns a PTO-protected linear DNA fragment comprising of the pyrE-BglII repair cassette obtained by PCR amplification from plasmid pF25-6brBglII with primers BLU001 (also designated as 001; sequence 5′-AGGTGGACTTTCAGGCCCTGCTATAAAGCC-3′) and BLU002 (also designated as 002; sequence 5′-AACTTGCAGGGACACTTTCTGGCGGAGAAC-3′) (FIG. 14). Both primers contain three PTO-modifications at their 5′ terminal end. This repair cassette was created using Q5 Hot Start High Fidelity DNA Polymerase (New England BioLabs) and was purified from the reaction mixture using the QIAquick PCR Purification kit (Qiagen).

The DNA templates were subsequently methylated using the purified methyltransferases EcMtORF02330 and EcMt2ORF02331. The expression and purification of the methyltransferases, the methylation of the DNA, and the purification of the methylated DNA from the methylation reactions were performed in the same way as described in Example 3.

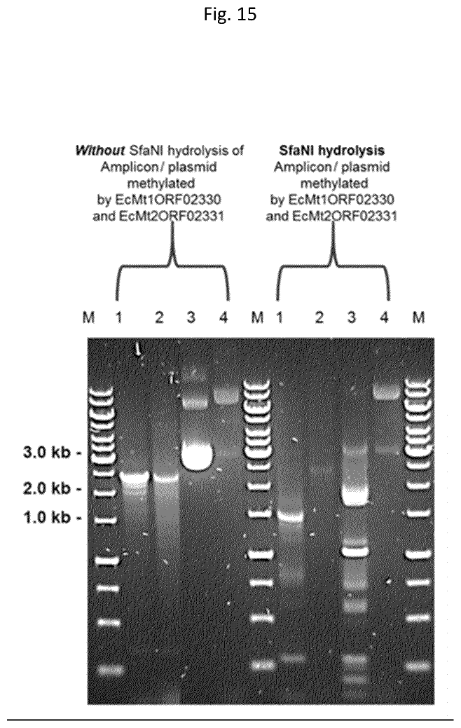

Next, the efficiency of the methylation reaction was checked by incubation of the methylated DNA and the original, unmethylated DNA with the restriction endonuclease SfaNI. SfaNI has the same recognition site (5′-GCATC(N)ₙ-3′) as the restriction enzymes of the type IIS RM system of Caldicellulosiruptor sp. DIB 104C strains and derivatives thereof (5′-GCATC-3′). In particular, 1 μg of DNA in 20 μl was incubated with 10 units of SfaNI (New England BioLabs) in the appropriate buffer for 2 hours at 37° C. The reaction products were then separated on a 0.7% agarose gel by electrophoresis, and the DNA was stained with SYBR Safe DNA Gel Stain (Invitrogen). The results show that the original, unmethylated pyrE-PCR amplicon (2,507 bps) and plasmid pF25-6brBglII (4,955 bps) are hydrolysed by the restriction enzyme SfaNI, while the methylated pyrE-PCR amplicon and plasmid pF25-6brBglII are not hydrolysed (FIG. 15).

Preparation of Electrocompetent Caldicellulosiruptor sp. DIB 104C pyrE M62 Cells and Transformation by Electroporation

With exception of the centrifugation steps and the pulse delivery for electroporation, all working steps for the preparation of electrocompetent cells and for the transformation were performed in the anaerobic workstation.

Electrocompetent cells of Caldicellulosiruptor sp. DIB 104C pyrE M62 were prepared according to Chung et al., 2000 (paragraph 3.2.2).

In total 50 μL of the electrocompetent cell suspension were transferred into a 1.5-mL reaction tube and mixed with 1 μg of either methylated or unmethylated DNA provided in 10 μL of water. After 15 minutes of incubation at room temperature, the cell suspensions were transferred into an ice-cold electroporation cuvette (1 mm) for electroporation. The electroporation parameters were as follows: single exponential pulse, field strength of 1 to 2 kV (10 to 20 V/cm), resistance of 500 to 700 Ohm, and capacity of 20 to 50 μF. Immediately after electroporation, 500 μL of LOD medium were transferred into the cuvette and the cell suspension was transferred into a Hungate tube containing 9 mL of LOD medium with 40 μM uracil. The culture was incubated overnight in an orbital shaker at 70° C. and 100 rpm.

### Selection of Transformants

After the overnight regeneration, the cells were collected by centrifugation for 20 minutes at 5,000×g and 22° C., and the cell pellet was resuspended in 1 mL of MOPS-buffered medium with CSM-Ura. Then, 100 μL of the cell suspension were transferred into a Hungate tube containing 9 mL of MOPS-buffered medium with filter paper and CSM-Ura for selection of prototrophic cells; another 100 μL were transferred into the same medium supplemented with yeast extract for control of cell viability. The cultures were subsequently incubated in an orbital shaker at 70° C. After 2 to 6 days of incubation, decomposition of the filter paper was observed for the cultures from the transformation with methylated DNA, while no decomposition was observed for the cultures from the transformation with unmethylated DNA. This indicates that methylation of template DNA with the methyltransferases EcMtORF02330 and EcMt2ORF02331 is essential for successful transformation of Caldicellulosiruptor sp. DIB 104C and derivatives thereof.

From the cultures showing decomposition of the filter paper, 30 μL were streaked for single cell colonies on solid LOD medium (without uracil). The plates were incubated for 4 to 6 days at 70° C. under anaerobic conditions until single cell colonies could be observed. These clones are expected to be uracil prototrophic.

Verification of the pyrE Genomic Integration by Restriction Analysis and Sanger Sequencing

Cells from seven single cell colonies (originating from the transformation with the methylated PTO-protected linear DNA fragment) were resuspended in 100 μL of MOPS-buffered medium with CSM-Ura and used to inoculate 9 mL of MOPS-buffered medium with filter paper and CSM-Ura. The cultures were incubated in an orbital shaker at 100 rpm and 70° C. until decomposition of the filter paper was observed. In total 100 μL of the latter cultures were transferred into 50 mL of LOD medium and incubated overnight at 100 rpm and 70° C. to obtain cell material for genomic DNA extraction. The extraction was performed using the MasterPure Gram Positive DNA Purification Kit from Epicentre according to the manufacturer's instructions.

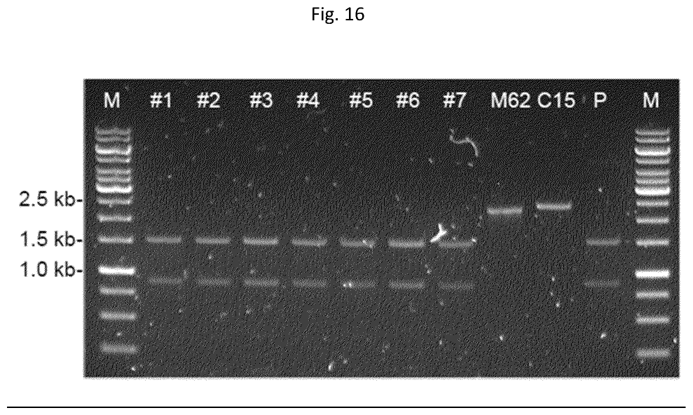

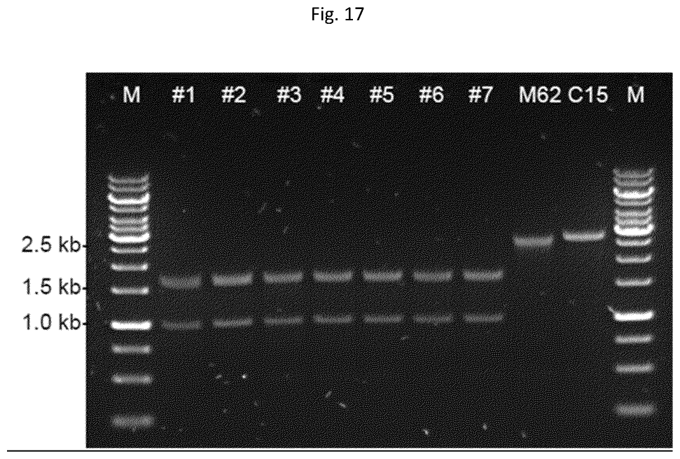

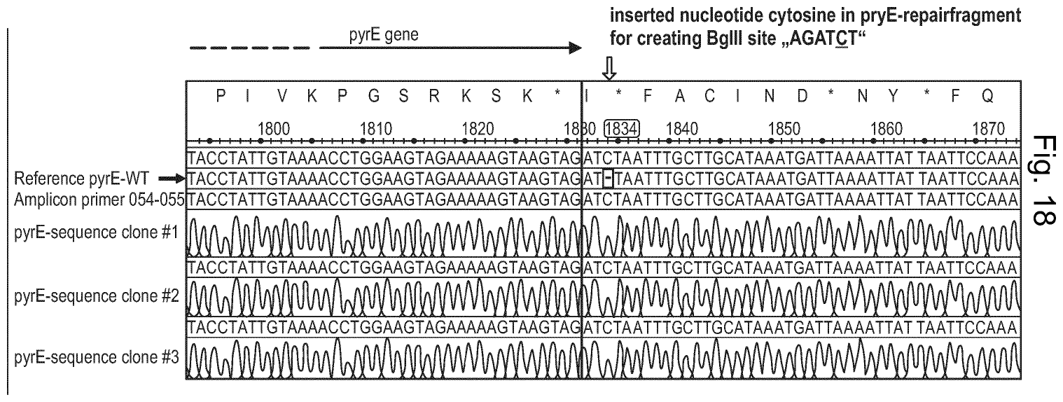

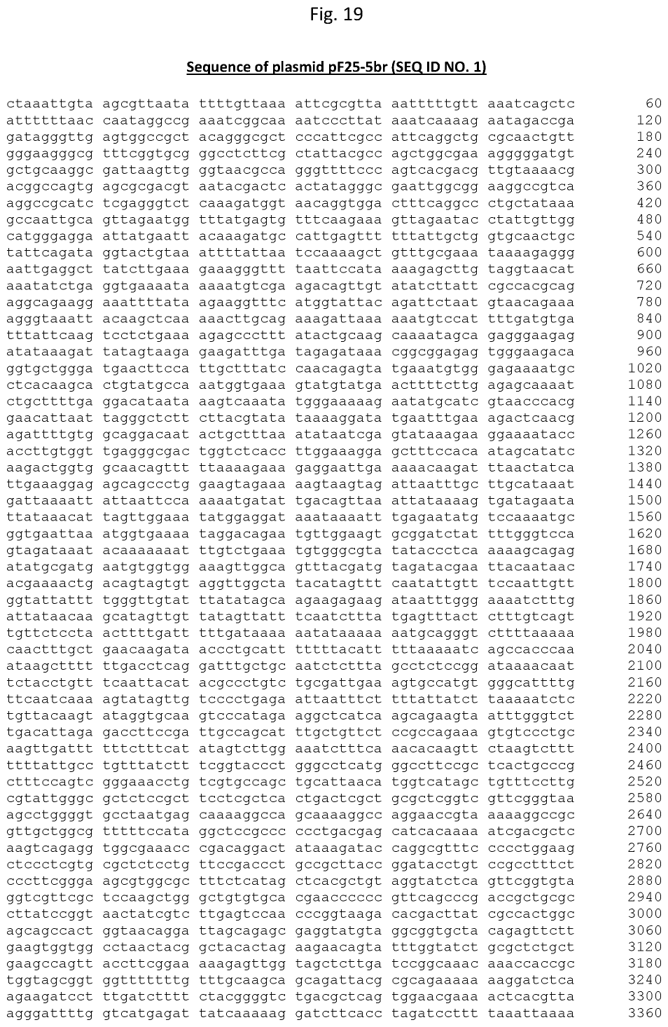

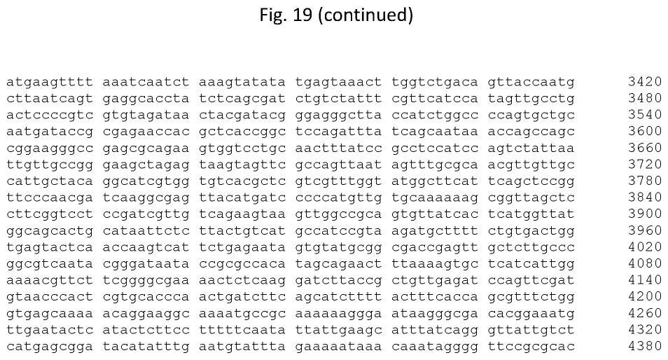

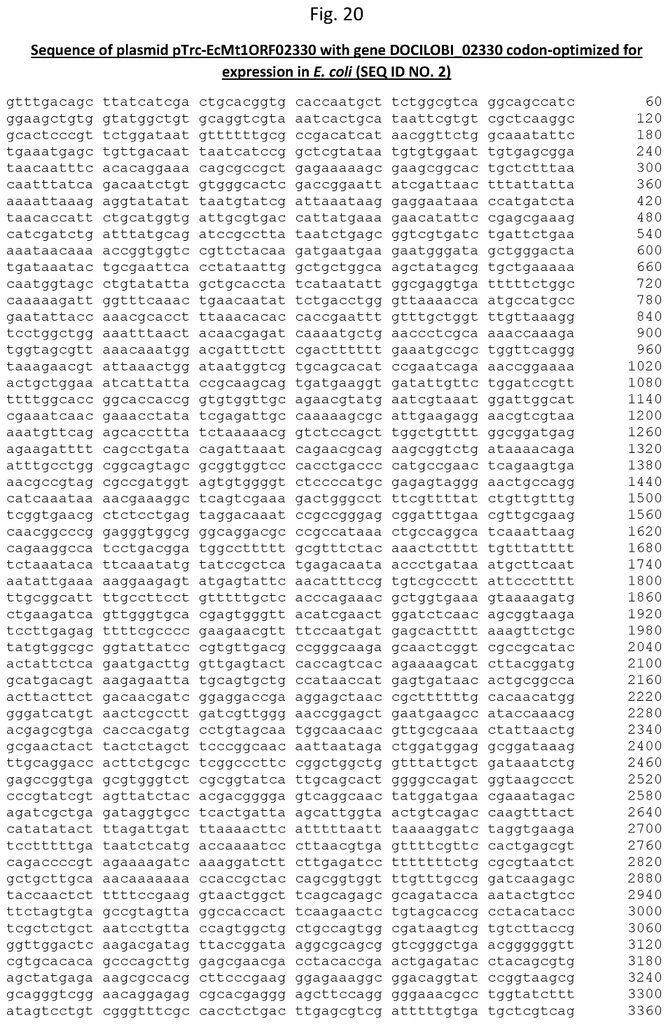

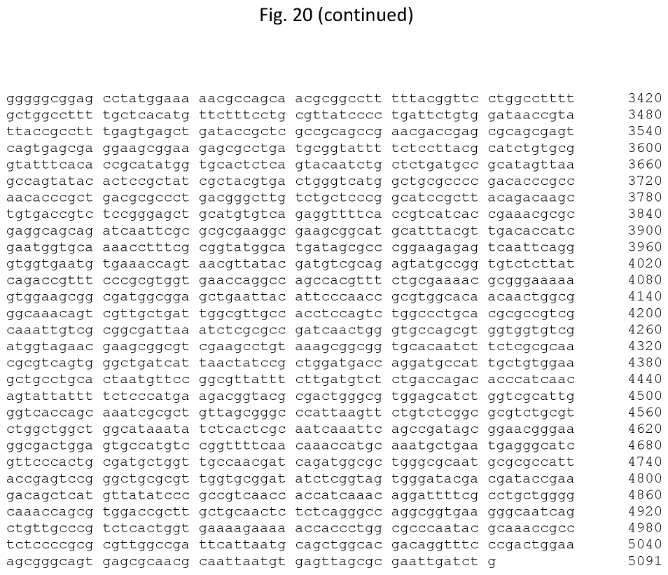

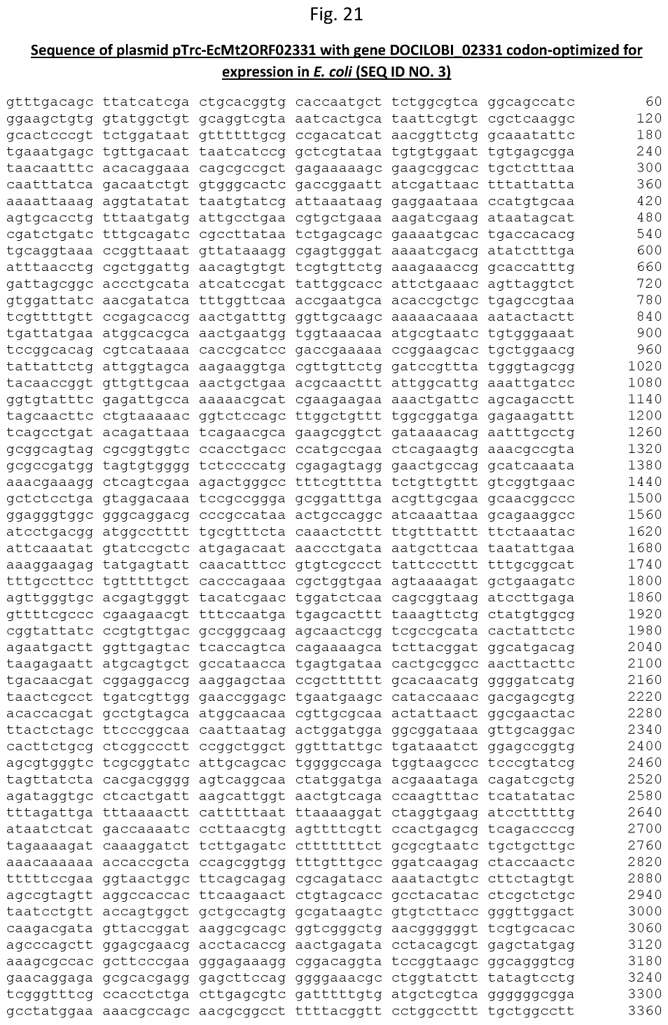

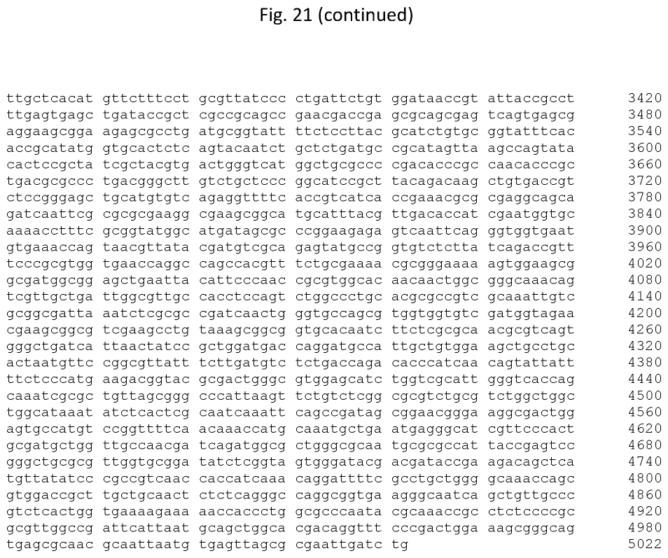

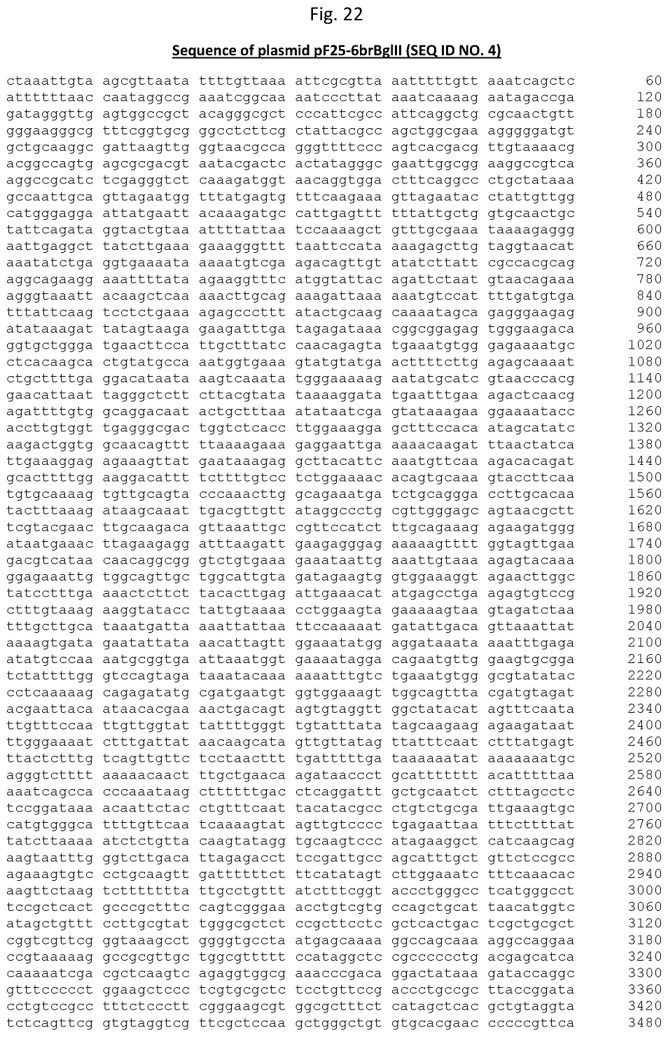

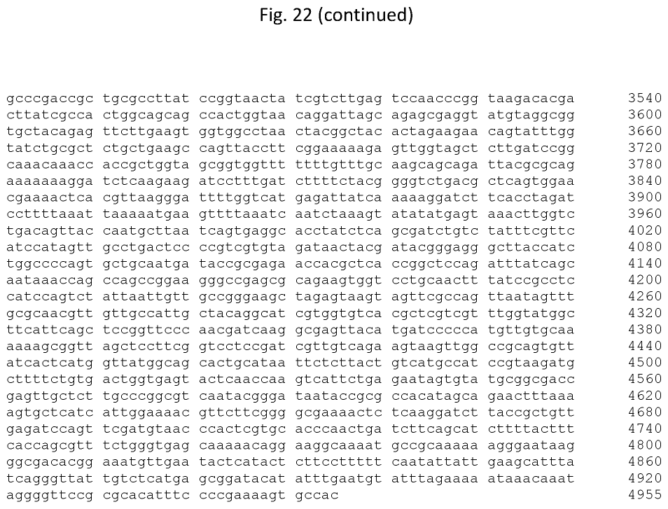

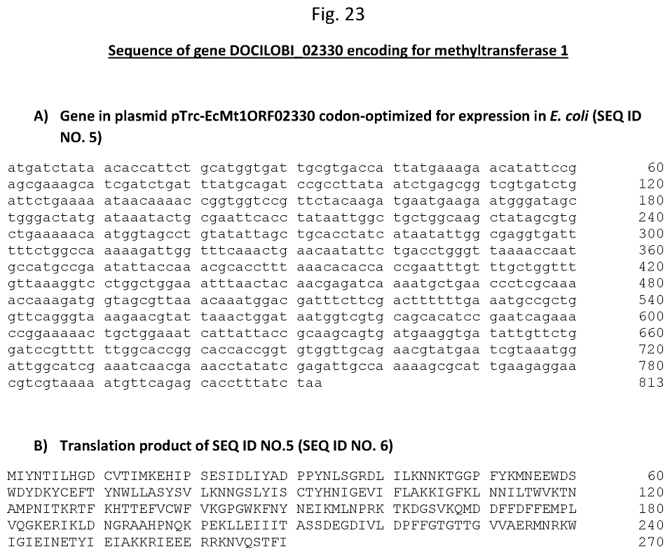

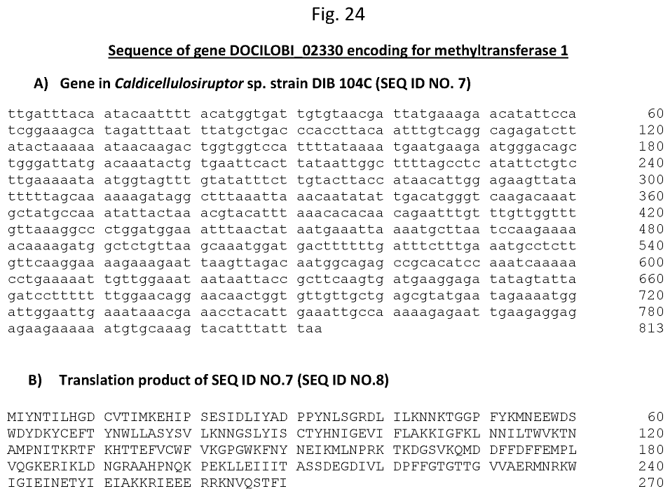

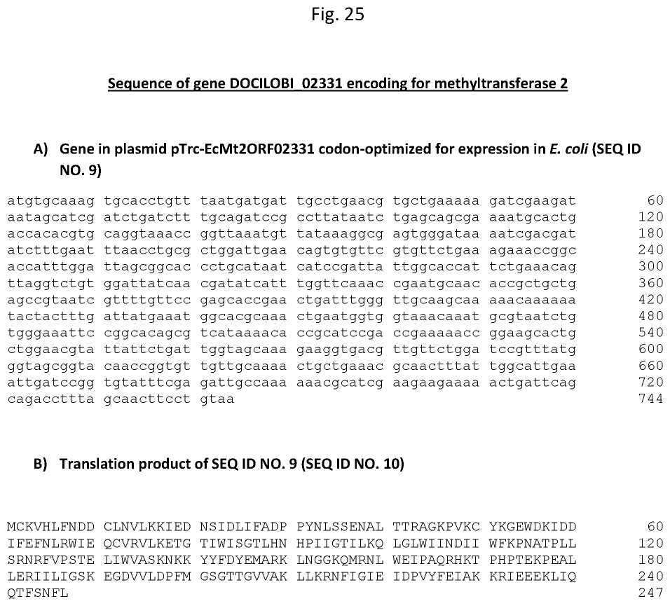

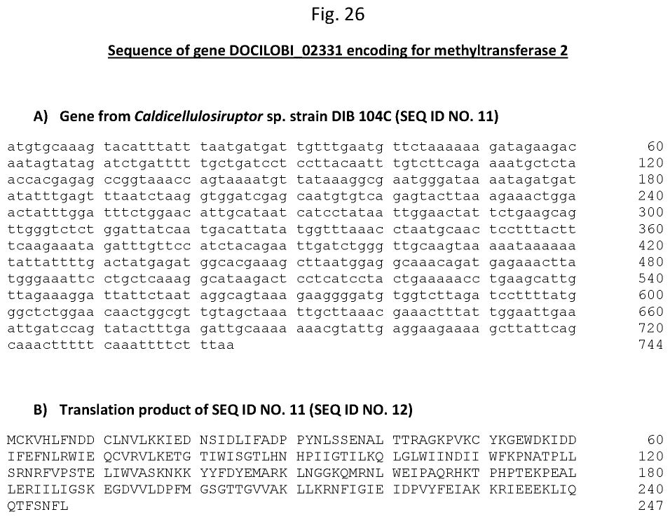

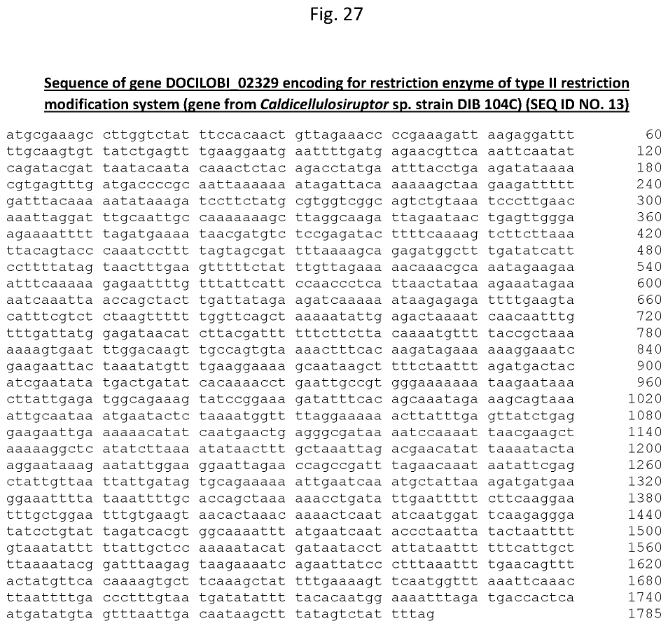

The site-specific genomic integration of the pyrE-BglII repair fragment in the seven isolated clones was confirmed by PCR and by Sanger sequencing. The PCR was performed on genomic DNA using primer pair BLU001 plus BLU002 and primer pair 054_pyrEup_f (sequence 5′-CTTGTCCGAACGTGAAAGAAGGTGGAATGG-3′) plus 055_pyrEdw_r (sequence 5′-TTGGCATTTCTCACGTGCCAGAAGGAAGAC-3′). As control, the same PCR was also performed on genomic DNA from strains Caldicellulosiruptor sp. DIB 104C (wild type) and Caldicellulosiruptor sp. DIB 104C pyrE M62 (pyrE knockout) and on plasmid pF25-6brBglII. As the site-specific integration of the repair fragment introduced a BglII recognition site that is not present in the Caldicellulosiruptor sp. DIB 104C wild type strain, correct integration was checked by cutting the PCR-amplified fragment using BglII (FIG. 16 and FIG. 17). The sizes of the expected fragments are given in Table 4 and Table 5. The results show the presence of the BglII recognition site in the anticipated position, thus confirming successful transformation of Caldicellulosiruptor sp. DIB 104C pyrE M62.

TABLE 4
Expected sizes of PCR amplicons and fragments
obtained by BglII hydrolysis.

| | Fragment obtained | Fragment(s) |
|---|---|---|
| | by PCR with primer | obtained by |
| DNA template | pair BLU001 plus BLU002 | BglII hydrolysis |
| 104C pyrE M62 (BglII) | 2507 bp | 927 bp + 1580 bp |
| 104C pyrE M62 | 2392 bp | 2392 bp |
| 104C (wildtype) | 2506 bp | 2506 bp |
| pF25-6brBglII | 2507 | 927 bp + 1580 bp |

TABLE 5
Expected sizes of PCR amplicons and fragments
obtained by BglII hydrolysis.

| | Fragment obtained | |
|---|---|---|
| | by PCR with primer | Fragment(s) |
| | pair 054_pyrEup_f | obtained by |
| DNA template | plus 055_pyrEdw_r | BglII hydrolysis |
| 104C pyrE M62 (BglII) | 2919 bp | 1089 bp + 1830 bp |
| 104C pyrE M62 | 2804 bp | 2804 bp |
| 104C (wildtype) | 2918 bp | 2918 bp |
| pF25-6brBglII | — | — |

Further proof for the correct genomic integration of the pyrE-BglII repair fragment was obtained by Sanger sequencing of the fragments obtained by PCR amplification with primer pair 054_pyrEup_f plus 055_pyrEdw_r from genomic DNA from clones #1, #2 and #3 (FIG. 18).

Alignment of the sequences with the pyrE wildtype sequence showed insertion of a cytosine in all tested clones resulting in the generation of the BglII recognition site “AGATCT”, which was not present in the pyrE wildtype strain. The identification of the BglII site immediately downstream of the pyrE gene by Sanger sequencing is an additional confirmation of the successful transformation event.

### CITED LITERATURE

Altschul S. F., Madden T. L., Schäffer A. A., Zhang J., Zhang Z., Miller W., Lipman D. J., 1997. Gapped BLAST and PSI-BLAST: a new generation of protein database search programs, Nucleic Acids Research. 25. 17. 3389-3402, https://doi.org/10.1093/nar/25.17.3389.
Chung D., Farkas J, Huddleston J. R. Olivar E., Westpheling J. 2012. Methylation by a Unique a-class N4-Cytosine Methyltransferase Is Required for DNA Transformation of Caldicellulosiruptor bescii DSM6725. PLoS ONE 7(8): e43844. doi:10.1371/journal.pone.0043844
Chung D., Farkas J. and Westpheling J. 2013. Overcoming restriction as a barrier to DNA transformation in Caldicellulosiruptor species results in efficient marker replacement. Biotechnology for Biofuels. 6:82.
Chung D, Cha M, Farkas J, Westpheling J. 2013. Construction of a stable replicating shuttle vector for Caldicellulosiruptor species: use for extending genetic methodologies to other members of this genus. Plos one. 8(5):e62881. DOI: 10.1371/journal.pone.0062881.
Chung D., Sarai N. S., Himmel M. E. and Bomble Y. E. 2020. Genetics of unstudied thermophiles for industry (chapter 2). In: Michael E. Himmel, Yannick J. Bomble (Eds.) Metabolic Pathway Engineering. Series: Methods in Molecular Biology. 1st ed. 2020. ISBN 978-1-0716-0194-5.
Farkas J., Chung D., Cha M., Copeland J., Grayeski P. and Westpheling J. 2013. Improved growth media and culture techniques for genetic analysis and assessment of biomass utilization by Caldicellulosiruptor bescii J Ind Microbiol Biotechnol 40, 41-49, DOI 10.1007/s10295-012-1202-1):
Gingeras, T. R. 1991. Restriction-modification systems: Genetic sentries and useful systems in the study of molecular genetics. In: Modern Microbial Genetics. 1st, ed. Vol. 1. Wiley-Liss, Inc, New York, 301-321.
Hungate, R. E. 1969. A roll tube method for cultivation of strict anaerobes. In: Methods in Microbiology Eds. Norris J R and Ribbons D W. pp 118-132. New York: Academic Press.
Mohapatra S. S., Biondi E. G. 2017. DNA Methylation in Prokaryotes: Regulation and Function. In: Krell T. (eds) Cellular Ecophysiology of Microbe. Handbook of Hydrocarbon and Lipid Microbiology. Springer, Cham. https://doi.org/10.1007/978-3-319-20796-4_23-1
Nelson M., Raschke, E., and McClelland, M. 1993. Effect of site-specific methylation on restriction endonucleases and DNA modification methyltransferases. Nucleic Acids Research. 21: 3139-3154.
Pingoud A., Wilson G. G. and Wende W. 2014. Type II restriction endonucleases—a historical perspective and more. Nucleic Acids Research. 42, 12: 7489-7527.
Roberts R. J., Tamas V. Posfai J. and Macelis D. 2010. REBASE—a database for DNA restriction and modification: enzymes, genes and genomes. Nucleic Acids Res. England. 38: D234-6. doi:10.1093/nar/gkp874. PMC 2808884. PMID 19846593.
Sambrook J. F. and Russel D. W. 2001. Molecular Cloning: A Laboratory Manual, 3rd ed., Volumes 1, 2 and 3. Cold Spring Harbor Laboratory Press. 2100 pp.
Svetlitchnyi V. A., Kensch O., Falkenhan D. A., Korseska S. G., Lippert N., Prinz M., Sassi J., Schickor A. and Curvers S. 2013. Single-step ethanol production from lignocellulose using novel extremely thermophilic bacteria. Biotechnology for Biofuels, 6: 31.
Tang P. W., Chua P. S, Chong S. K., Mohamed M. S., Choon Y. W., Deris S., Omatu S., Corchado J. M. Chan W. H.-and Rahin R. A. 2015. Recent Patents on Biotechnology. 9. 3.
Westphal L. L., Sauvey, P., Champion, M. M.: Ehrenreich, I. M.; Finkel, S. E. 2016. Genomewide Dam methylation in Escherichia coli during long-term stationary phase. mSystems 1(6):e00130-16. doi:10.1128/mSystems.00130-16.
Wilson G. G., Wang, H., Heiter D. F. 2012. Restriction Enzymes in Microbiology, Biotechnology and Biochemistry. Encuentro No. 93: 19-48.

1. An enzyme composition comprising at least two different thermostable polypeptides having type II DNA methyltransferase activity, wherein
a) the first polypeptide is a thermostable polypeptide having type II DNA methyltransferase activity, wherein said polypeptide methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, and
b) the second polypeptide is a thermostable polypeptide having type II DNA methyltransferase activity, wherein said polypeptide methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

2. The enzyme composition according to claim 1, wherein the methylation is a N6-methyladenine modification and the thermostable polypeptides are N6 adenine methylases.

3. The enzyme composition of claim 1, wherein the first polypeptide comprises the amino acid sequence of SEQ ID NO: 6 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 6, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

4. The enzyme composition of claim 1, wherein the first polypeptide comprises the amino acid sequence of SEQ ID NO: 8 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

5. The enzyme composition of claim 1, wherein the second polypeptide comprises the amino acid sequence of SEQ ID NO: 10 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 10, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

6. The enzyme composition of claim 1, wherein the second polypeptide comprises the amino acid sequence of SEQ ID NO: 12 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

7. The enzyme composition of claim 1, wherein the first thermostable polypeptide comprises
the amino acid sequence of SEQ ID NO: 6 or SEQ ID NO: 8, or variants of SEQ ID NO: 6 or SEQ ID NO: 8, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 6 or SEQ ID NO: 8, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′, and the second thermostable polypeptide comprises
the amino acid sequence of SEQ ID NO: 10 or SEQ ID NO: 12, or variants of SEQ ID NO: 10 or SEQ ID NO: 12, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO: 10 or SEQ ID NO: 12, and wherein said variant(s) methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

8. A nucleic acid molecule encoding a polypeptide according to claim 1.

9. The nucleic acid molecule of claim 8, wherein the nucleic acid molecule comprises a nucleic acid sequence selected from the group consisting of SEQ ID NO.5, SEQ ID NO. 7, SEQ ID NO. 9 and SEQ ID NO. 11 or variants thereof, wherein the nucleic acid sequence of said variants comprising at least a minimum percentage sequence identity of at least 85%, at least 90%, at least 93%, at least 96%, at least 97%, at least 98% or at least 99% to the nucleic acid sequence of SEQ ID NO.5, SEQ ID NO. 7, SEQ ID NO. 9 and SEQ ID NO. 11, and wherein said variant(s) encodes a thermostable polypeptide methylate an adenine in a asymmetric DNA recognition site, where the adenine nucleotide is followed by a thymine nucleotide in the linear sequence of bases along its 5′→3′ direction, wherein the DNA recognition site is 5′-GCATC-3′ and/or wherein said polypeptide methylate the adenine in a complement DNA recognition site, where a thymine nucleotide is followed by a adenine nucleotide in the linear sequence of bases along its 3′→5′ direction, wherein the DNA recognition site is 3′-CGTAG-5′.

10. The nucleic acid molecule of claim 8, wherein the nucleic acid molecule comprises the nucleic acid sequence of SEQ ID NO. 13 or variants thereof, wherein the nucleic acid sequence of said variants comprising at least a minimum percentage sequence identity of at least 85%, at least 90%, at least 93%, at least 96%, at least 97%, at least 98% or at least 99% to the nucleic acid sequence of SEQ ID NO.5, SEQ ID NO. 7, SEQ ID NO. 9 and SEQ ID NO. 11, and wherein said variant(s) encodes a polypeptide having restriction endonuclease activity, wherein the DNA recognition site of said polypeptide is 5′-GCATC-3′ and/or 3′-CGTAG-5′.

11. A vector comprising a nucleic acid molecule according to claim 8.

12. The vector according to claim 11, wherein the vector comprises the sequence of SEQ ID NO. 2 and/or SEQ ID NO 3.

13. A host cell transformed, transduced or transfected with a vector according to claim 11.

14. A restriction modification system comprising an enzyme composition of claim 1 and a polypeptide having restriction endonuclease activity, wherein the DNA recognition site of said restriction endonuclease is 5′-GCATC-3′ and/or 3′-CGTAG-5′.

15. The restriction modification system of claim 14, wherein the restriction endonuclease is encoded by the nucleic acid sequence of SEQ ID NO: 13 or variants thereof, wherein the amino acid sequence of said variants comprising at least a minimum percentage sequence identity of at least 85%, at least 90%, at least 93%, at least 96%, at least 97%, at least 98% or at least 99% to the amino acid sequence of SEQ ID NO. 13, wherein the DNA recognition site of said variant(s) is 5′-GCATC-3′ and/or 3′-CGTAG-5′.

16. A method for the in vitro methylation of DNA by using an enzyme composition of claim 1.

17. A method for introducing an exogenous DNA molecule into a target bacterium, comprising steps of:
1) co-expression of an enzyme composition comprising at least two different thermostable polypeptides having type II DNA methyltransferase activity according to claim 1 in a microorganism;
2) introducing an exogenous target DNA molecule into said microorganism to obtain an exogenous target DNA molecule methylated by said polypeptides having methyltransferase activity; and
3) introducing said methylated exogenous target DNA molecule into the target bacterium.

18. The method according to claim 17, wherein the target bacterium is a bacterium of the species Caldicellulosiruptor saccharolyticus, Caldicellulosiruptor changbaiensis, Caldicellulosiruptor naganoensis or the species or strain Caldicellulosiruptor sp. E32.

19. The method according to claim 17, wherein the target bacterium is an isolated bacterium of the genus Caldicellulosiruptor sp., wherein the bacterium is selected from the group consisting of Caldicellulosiruptor sp. BluConL70 having the DSMZ Accession number 33496, Caldicellulosiruptor sp. BluConL60 having the DSMZ Accession number 33252, Caldicellulosiruptor sp. BluCon085 having the DSMZ Accession number 33485 Caldicellulosiruptor sp. BluCon052 having the DSMZ Accession number 33470, Caldicellulosiruptor sp. BluCon006 having the DSMZ Accession number 33095, Caldicellulosiruptor sp. BluCon014 (DSMZ Accession number 33096) and Caldicellulosiruptor sp. BluCon016 (DSMZ Accession number 33097), microorganism derived therefrom, progenies or mutants thereof, wherein the mutants thereof retaining the properties of BluConL70, BluConL60, BluCon085, BluCon052, BluCon006, BluCon014 and/or BluCon016.

20. The method according to claim 17, wherein the target bacterium is an isolated bacterium of the genus Caldicellulosiruptor sp., wherein the bacterium is a microorganism of the genus Caldicellulosiruptor is selected from the group consisting of Caldicellulosiruptor sp. DIB 041C (DSMZ Accession number 25771), Caldicellulosiruptor sp. DIB 004C (DSMZ Accession number 25177), Caldicellulosiruptor sp. DIB 101C (DSMZ Accession number 25178), Caldicellulosiruptor sp. DIB 103C (DSMZ Accession number 25773), Caldicellulosiruptor sp. DIB 107C (DSMZ Accession number 25775), Caldicellulosiruptor sp. DIB 087C (DSMZ Accession number 25772), Caldicellulosiruptor sp. DIB 104C (DSMZ Accession number 25774), Caldicellulosiruptor sp. BluCon006 (DSMZ Accession number 33095), Caldicellulosiruptor sp. BluCon014 (DSMZ Accession number 33096), Caldicellulosiruptor sp. BluCon016 (DSMZ Accession number 33097) and Caldicellulosiruptor sp. BluConL60 (DSMZ Accession number 33252).

21. The method according to claim 17, wherein the target bacterium is Caldicellulosiruptor sp. DIB 104C (DSMZ Accession number 25774) or Caldicellulosiruptor sp. BluCon085 (DSMZ Accession number 33485).

22. The method according to claim 17, wherein the target bacterium is an isolated bacterium of the genus Caldicellulosiruptor saccharolyticus (DSMZ Accession number 8903) and Caldicellulosiruptor changbaiensis (DSMZ Accession number 26941), Caldicellulosiruptor naganoensis and the species or strain Caldicellulosiruptor sp. E32.

23. A method for introducing an exogenous DNA molecule into a target bacterium of the species Caldicellulosiruptor saccharolyticus, Caldicellulosiruptor changbaiensis, Caldicellulosiruptor naganoensis or the species or strain Caldicellulosiruptor sp. E32 or of the genus Caldicellulosiruptor sp., wherein a polypeptide having restriction endonuclease activity defined in claim 14 is inhibited by an inhibitor in the bacteria and/or the gene encoding said polypeptide is knocked-out, wherein said inhibitor inhibits the expression of said polypeptide and/or binds to a protein product of a gene coding said polypeptide.

24. The method according to claim 24, wherein the bacterium is an isolated bacterium of the genus Caldicellulosiruptor sp., wherein the bacterium is selected from the group consisting of Caldicellulosiruptor sp. BluConL70 having the DSMZ Accession number 33496, Caldicellulosiruptor sp. BluConL60 having the DSMZ Accession number 33252, Caldicellulosiruptor sp. BluCon085 having the DSMZ Accession number 33485 Caldicellulosiruptor sp. BluCon052 having the DSMZ Accession number 33470, Caldicellulosiruptor sp. BluCon006 having the DSMZ Accession number 33095, Caldicellulosiruptor sp. BluCon014 (DSMZ Accession number 33096) and Caldicellulosiruptor sp. BluCon016 (DSMZ Accession number 33097), microorganism derived therefrom, progenies or mutants thereof, wherein the mutants thereof retaining the properties of BluConL70, BluConL60, BluCon085, BluCon052, BluCon006, BluCon014 and/or BluCon016.

25. The method according to claim 24, wherein the bacterium is an isolated bacterium of the genus Caldicellulosiruptor sp., wherein the bacterium is a microorganism of the genus Caldicellulosiruptor is selected from the group consisting of Caldicellulosiruptor sp. DIB 041C (DSMZ Accession number 25771), Caldicellulosiruptor sp. DIB 004C (DSMZ Accession number 25177), Caldicellulosiruptor sp. DIB 101C (DSMZ Accession number 25178), Caldicellulosiruptor sp. DIB 103C (DSMZ Accession number 25773), Caldicellulosiruptor sp. DIB 107C (DSMZ Accession number 25775), Caldicellulosiruptor sp. DIB 087C (DSMZ Accession number 25772), Caldicellulosiruptor sp. DIB 104C (DSMZ Accession number 25774), Caldicellulosiruptor sp. BluCon006 (DSMZ Accession number 33095), Caldicellulosiruptor sp. BluCon014 (DSMZ Accession number 33096), Caldicellulosiruptor sp. BluCon016 (DSMZ Accession number 33097) and Caldicellulosiruptor sp. BluConL60 (DSMZ Accession number 33252).

26. The method according to claim 24, wherein the target bacterium is Caldicellulosiruptor sp. DIB 104C (DSMZ Accession number 25774) or Caldicellulosiruptor sp. BluCon085 (DSMZ Accession number 33485).

27. A host cell, characterized in that a polypeptide having restriction endonuclease activity defined in claim 14 is inhibited by an inhibitor in the host cell and/or the gene encoding said polypeptide is knocked-out in the host cell, wherein said inhibitor inhibits the expression of said polypeptide and/or binds to a protein product of a gene coding said polypeptide.

28. The host cell according to claim 27, wherein the host cell is a bacterium of the species Caldicellulosiruptor saccharolyticus, Caldicellulosiruptor changbaiensis, Caldicellulosiruptor naganoensis and the species or strain Caldicellulosiruptor sp. E32.

29. The host cell according to claim 27, wherein the host cell is an isolated bacterium of the genus Caldicellulosiruptor sp., wherein the bacterium is selected from the group consisting of Caldicellulosiruptor sp. BluConL70 having the DSMZ Accession number 33496, Caldicellulosiruptor sp. BluConL60 having the DSMZ Accession number 33252, Caldicellulosiruptor sp. BluCon085 having the DSMZ Accession number 33485 Caldicellulosiruptor sp. BluCon052 having the DSMZ Accession number 33470, Caldicellulosiruptor sp. BluCon006 having the DSMZ Accession number 33095, Caldicellulosiruptor sp. BluCon014 (DSMZ Accession number 33096) and Caldicellulosiruptor sp. BluCon016 (DSMZ Accession number 33097), microorganism derived therefrom, progenies or mutants thereof, wherein the mutants thereof retaining the properties of BluConL70, BluConL60, BluCon085, BluCon052, BluCon006, BluCon014 and/or BluCon016.

30. The host cell according to claim 27, wherein the host cell is an isolated bacterium of the genus Caldicellulosiruptor sp., wherein the bacterium is a microorganism of the genus Caldicellulosiruptor is selected from the group consisting of Caldicellulosiruptor sp. DIB 041C (DSMZ Accession number 25771), Caldicellulosiruptor sp. DIB 004C (DSMZ Accession number 25177), Caldicellulosiruptor sp. DIB 101C (DSMZ Accession number 25178), Caldicellulosiruptor sp. DIB 103C (DSMZ Accession number 25773), Caldicellulosiruptor sp. DIB 107C (DSMZ Accession number 25775), Caldicellulosiruptor sp. DIB 087C (DSMZ Accession number 25772), Caldicellulosiruptor sp. DIB 104C (DSMZ Accession number 25774), Caldicellulosiruptor sp. BluCon006 (DSMZ Accession number 33095), Caldicellulosiruptor sp. BluCon014 (DSMZ Accession number 33096), Caldicellulosiruptor sp. BluCon016 (DSMZ Accession number 33097) and Caldicellulosiruptor sp. BluConL60 (DSMZ Accession number 33252).

31. The host cell according to claim 27, wherein the host cell is Caldicellulosiruptor sp. DIB 104C (DSMZ Accession number 25774) or Caldicellulosiruptor sp. BluCon085 (DSMZ Accession number 33485).


SEQUENCE LISTING (39 sequences)

SEQ ID NO: 1 - DNA, 4380 bp, Artificial Sequence
  plasmid pF25-5br (SEQ ID NO. 1)
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatctcgagggtctcaaagatggtaacaggtggactttcaggccctgctataaagccaattgcagttagaatggtttatgagtgtttcaagaaagttagaatacctattgttggcatgggaggaattatgaattacaaagatgccattgagttttttattgctggtgcaactgctattcagataggtactgtaaattttattaatccaaaagctgtttgcgaaataaaagagggaattgaggcttatcttgaaagaaagggttttaattccataaaagagcttgtaggtaacataaatatctgaggtgaaaataaaaatgtcgaagacagttgtatatcttattcgccacgcagaggcagaaggaaattttataagaaggtttcatggtattacagattctaatgtaacagaaaagggtaaattacaagctcaaaaacttgcagaaagattaaaaaatgtccattttgatgtgatttattcaagtcctctgaaaagagccctttatactgcaagcaaaatagcagagggaagagatataaagattatagtaagagaagatttgatagagataaacggcggagagtgggaagacaggtgctgggatgaacttccattgctttatccaacagagtatgaaatgtgggagaaaatgcctcacaagcactgtatgccaaatggtgaaagtatgtatgaacttttcttgagagcaaaatctgcttttgaggacataataaagtcaaatatgggaaaaagaatatgcatcgtaacccacggaacattaattagggctcttcttacgtatataaaaggatatgaatttgaaagactcaacgagattttgtggcaggacaatactgctttaaatataatcgagtataaagaaggaaaataccaccttgtggttgagggcgactggtctcaccttggaaaggagctttccacaatagcatatcaagactggtggcaacagtttttaaaagaaagaggaattgaaaaacaagatttaactatcattgaaaggagagcagccctggaagtagaaaaagtaagtagattaatttgcttgcataaatgattaaaattattaattccaaaaatgatattgacagttaaattataaaagtgatagaatattataaacattagttggaaatatggaggataaataaaatttgagaatatgtccaaaatgcggtgaattaaatggtgaaaataggacagaatgttggaagtgcggatctattttgggtccagtagataaatacaaaaaaatttgtctgaaatgtgggcgtatataccctcaaaaagcagagatatgcgatgaatgtggtggaaagttggcagtttacgatgtagatacgaattacaataacacgaaaactgacagtagtgtaggttggctatacatagtttcaatattgtttccaattgttggtattattttgggttgtatttatatagcaagaagagaagataatttgggaaaatctttgattataacaagcatagttgttatagttatttcaatctttatgagtttactctttgtcagttgttctcctaacttttgatttttgataaaaaatataaaaaaatgcagggtcttttaaaaacaactttgctgaacaagataaccctgcatttttttacatttttaaaaatcagccacccaaataagcttttttgacctcaggatttgctgcaatctctttagcctctccggataaaacaattctacctgtttcaattacatacgccctgtctgcgattgaaagtgccatgtgggcattttgttcaatcaaaagtatagttgtcccctgagaattaatttcttttattatcttaaaaatctctgttacaagtataggtgcaagtcccatagaaggctcatcaagcagaagtaatttgggtcttgacattagagaccttccgattgccagcatttgctgttctccgccagaaagtgtccctgcaagttgattttttctttcatatagtcttggaaatctttcaaacacaagttctaagtctttttttattgcctgtttatctttcggtaccctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcac

SEQ ID NO: 2 - DNA, 5091 bp, Artificial Sequence
  plasmid pTrc-EcMt1ORF02330 with gene DOCILOBI_02330 codon-optimized for expression in E. coli (SEQ ID NO. 2)
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgatctataacaccattctgcatggtgattgcgtgaccattatgaaagaacatattccgagcgaaagcatcgatctgatttatgcagatccgccttataatctgagcggtcgtgatctgattctgaaaaataacaaaaccggtggtccgttctacaagatgaatgaagaatgggatagctgggactatgataaatactgcgaattcacctataattggctgctggcaagctatagcgtgctgaaaaacaatggtagcctgtatattagctgcacctatcataatattggcgaggtgatttttctggccaaaaagattggtttcaaactgaacaatattctgacctgggttaaaaccaatgccatgccgaatattaccaaacgcacctttaaacacaccaccgaatttgtttgctggtttgttaaaggtcctggctggaaatttaactacaacgagatcaaaatgctgaaccctcgcaaaaccaaagatggtagcgttaaacaaatggacgatttcttcgacttttttgaaatgccgctggttcagggtaaagaacgtattaaactggataatggtcgtgcagcacatccgaatcagaaaccggaaaaactgctggaaatcattattaccgcaagcagtgatgaaggtgatattgttctggatccgttttttggcaccggcaccaccggtgtggttgcagaacgtatgaatcgtaaatggattggcatcgaaatcaacgaaacctatatcgagattgccaaaaagcgcattgaagaggaacgtcgtaaaaatgttcagagcacctttatctaaaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 3 - DNA, 5022 bp, Artificial Sequence
  Sequence of plasmid pTrc-EcMt2ORF02331 with gene DOCILOBI_02331 codon-optimized for expression in E. coli (SEQ ID NO. 3)
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgcaaagtgcacctgtttaatgatgattgcctgaacgtgctgaaaaagatcgaagataatagcatcgatctgatctttgcagatccgccttataatctgagcagcgaaaatgcactgaccacacgtgcaggtaaaccggttaaatgttataaaggcgagtgggataaaatcgacgatatctttgaatttaacctgcgctggattgaacagtgtgttcgtgttctgaaagaaaccggcaccatttggattagcggcaccctgcataatcatccgattattggcaccattctgaaacagttaggtctgtggattatcaacgatatcatttggttcaaaccgaatgcaacaccgctgctgagccgtaatcgttttgttccgagcaccgaactgatttgggttgcaagcaaaaacaaaaaatactactttgattatgaaatggcacgcaaactgaatggtggtaaacaaatgcgtaatctgtgggaaattccggcacagcgtcataaaacaccgcatccgaccgaaaaaccggaagcactgctggaacgtattattctgattggtagcaaagaaggtgacgttgttctggatccgtttatgggtagcggtacaaccggtgttgttgcaaaactgctgaaacgcaactttattggcattgaaattgatccggtgtatttcgagattgccaaaaaacgcatcgaagaagaaaaactgattcagcagacctttagcaacttcctgtaaaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 4 - DNA, 4955 bp, Artificial Sequence
  plasmid pF25-6brBglII (SEQ ID NO. 4)
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatctcgagggtctcaaagatggtaacaggtggactttcaggccctgctataaagccaattgcagttagaatggtttatgagtgtttcaagaaagttagaatacctattgttggcatgggaggaattatgaattacaaagatgccattgagttttttattgctggtgcaactgctattcagataggtactgtaaattttattaatccaaaagctgtttgcgaaataaaagagggaattgaggcttatcttgaaagaaagggttttaattccataaaagagcttgtaggtaacataaatatctgaggtgaaaataaaaatgtcgaagacagttgtatatcttattcgccacgcagaggcagaaggaaattttataagaaggtttcatggtattacagattctaatgtaacagaaaagggtaaattacaagctcaaaaacttgcagaaagattaaaaaatgtccattttgatgtgatttattcaagtcctctgaaaagagccctttatactgcaagcaaaatagcagagggaagagatataaagattatagtaagagaagatttgatagagataaacggcggagagtgggaagacaggtgctgggatgaacttccattgctttatccaacagagtatgaaatgtgggagaaaatgcctcacaagcactgtatgccaaatggtgaaagtatgtatgaacttttcttgagagcaaaatctgcttttgaggacataataaagtcaaatatgggaaaaagaatatgcatcgtaacccacggaacattaattagggctcttcttacgtatataaaaggatatgaatttgaaagactcaacgagattttgtggcaggacaatactgctttaaatataatcgagtataaagaaggaaaataccaccttgtggttgagggcgactggtctcaccttggaaaggagctttccacaatagcatatcaagactggtggcaacagtttttaaaagaaagaggaattgaaaaacaagatttaactatcattgaaaggagagaaagttatgaataaagaggcttacattcaaatgttcaaagacacagatgcacttttggaaggacattttcttttgtcctctggaaaacacagtgcaaagtaccttcaatgtgcaaaagtgttgcagtacccaaacttggcagaaatgatctgcagggaccttgcacaatactttaaagataagcaaattgacgttgttataggccctgcgttgggagcagtaacgctttcgtacgaacttgcaagacagttaaattgccgttccatctttgcagaaagagaagatgggataatgaaacttagaagaggatttaagattgaagagggagaaaaagttttggtagttgaagacgtcataacaacaggcgggtctgtgaaagaaataattgaaattgtaaaagagtacaaaggagaaattgtggcagttgctggcattgtagatagaagtggtggaaaggtagaacttggctatcctttgaaaactcttcttacacttgagattgaaacatatgagcctgaagagtgtccgctttgtaaagaaggtatacctattgtaaaacctggaagtagaaaaagtaagtagatctaatttgcttgcataaatgattaaaattattaattccaaaaatgatattgacagttaaattataaaagtgatagaatattataaacattagttggaaatatggaggataaataaaatttgagaatatgtccaaaatgcggtgaattaaatggtgaaaataggacagaatgttggaagtgcggatctattttgggtccagtagataaatacaaaaaaatttgtctgaaatgtgggcgtatataccctcaaaaagcagagatatgcgatgaatgtggtggaaagttggcagtttacgatgtagatacgaattacaataacacgaaaactgacagtagtgtaggttggctatacatagtttcaatattgtttccaattgttggtattattttgggttgtatttatatagcaagaagagaagataatttgggaaaatctttgattataacaagcatagttgttatagttatttcaatctttatgagtttactctttgtcagttgttctcctaacttttgatttttgataaaaaatataaaaaaatgcagggtcttttaaaaacaactttgctgaacaagataaccctgcatttttttacatttttaaaaatcagccacccaaataagcttttttgacctcaggatttgctgcaatctctttagcctctccggataaaacaattctacctgtttcaattacatacgccctgtctgcgattgaaagtgccatgtgggcattttgttcaatcaaaagtatagttgtcccctgagaattaatttcttttattatcttaaaaatctctgttacaagtataggtgcaagtcccatagaaggctcatcaagcagaagtaatttgggtcttgacattagagaccttccgattgccagcatttgctgttctccgccagaaagtgtccctgcaagttgattttttctttcatatagtcttggaaatctttcaaacacaagttctaagtctttttttattgcctgtttatctttcggtaccctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 5 - DNA, 813 bp, Artificial Sequence
  plasmid pTrc-EcMt1ORF02330 codon-optimized for expression in E. coli (SEQ ID NO. 5)
atgatctataacaccattctgcatggtgattgcgtgaccattatgaaagaacatattccgagcgaaagcatcgatctgatttatgcagatccgccttataatctgagcggtcgtgatctgattctgaaaaataacaaaaccggtggtccgttctacaagatgaatgaagaatgggatagctgggactatgataaatactgcgaattcacctataattggctgctggcaagctatagcgtgctgaaaaacaatggtagcctgtatattagctgcacctatcataatattggcgaggtgatttttctggccaaaaagattggtttcaaactgaacaatattctgacctgggttaaaaccaatgccatgccgaatattaccaaacgcacctttaaacacaccaccgaatttgtttgctggtttgttaaaggtcctggctggaaatttaactacaacgagatcaaaatgctgaaccctcgcaaaaccaaagatggtagcgttaaacaaatggacgatttcttcgacttttttgaaatgccgctggttcagggtaaagaacgtattaaactggataatggtcgtgcagcacatccgaatcagaaaccggaaaaactgctggaaatcattattaccgcaagcagtgatgaaggtgatattgttctggatccgttttttggcaccggcaccaccggtgtggttgcagaacgtatgaatcgtaaatggattggcatcgaaatcaacgaaacctatatcgagattgccaaaaagcgcattgaagaggaacgtcgtaaaaatgttcagagcacctttatctaa

SEQ ID NO: 6 - protein, 270 aa, Artificial Sequence
  Translation product of SEQ ID NO.5 (SEQ ID NO. 6)
MIYNTILHGDCVTIMKEHIPSESIDLIYADPPYNLSGRDLILKNNKTGGPFYKMNEEWDSWDYDKYCEFTYNWLLASYSVLKNNGSLYISCTYHNIGEVIFLAKKIGFKLNNILTWVKTNAMPNITKRTFKHTTEFVCWFVKGPGWKFNYNEIKMLNPRKTKDGSVKQMDDFFDFFEMPLVQGKERIKLDNGRAAHPNQKPEKLLEIIITASSDEGDIVLDPFFGTGTTGVVAERMNRKWIGIEINETYIEIAKKRIEEERRKNVQSTFI

SEQ ID NO: 7 - DNA, 813 bp, Artificial Sequence
  Gene in Caldicellulosiruptor sp. strain DIB 104C (SEQ ID NO. 7)
ttgatttacaatacaattttacatggtgattgtgtaacgattatgaaagaacatattccatcggaaagcatagatttaatttatgctgacccaccttacaatttgtcaggcagagatcttatactaaaaaataacaagactggtggtccattttataaaatgaatgaagaatgggacagctgggattatgacaaatactgtgaattcacttataattggcttttagcctcatattctgtcttgaaaaataatggtagtttgtatatttcttgtacttaccataacattggagaagttatatttttagcaaaaaagataggctttaaattaaacaatatattgacatgggtcaagacaaatgctatgccaaatattactaaacgtacatttaaacacacaacagaatttgtttgttggtttgttaaaggccctggatggaaatttaactataatgaaattaaaatgcttaatccaagaaaaacaaaagatggctctgttaagcaaatggatgacttttttgatttctttgaaatgcctcttgttcaaggaaaagaaagaattaagttagacaatggcagagccgcacatccaaatcaaaaacctgaaaaattgttggaaataataattaccgcttcaagtgatgaaggagatatagtattagatcctttttttggaacaggaacaactggtgttgttgctgagcgtatgaatagaaaatggattggaattgaaataaacgaaacctacattgaaattgccaaaaagagaattgaagaggagagaagaaaaaatgtgcaaagtacatttatttaa

SEQ ID NO: 8 - protein, 270 aa, Artificial Sequence; identical to SEQ ID NO: 6
  Translation product of SEQ ID NO.7 (SEQ ID NO.8)

SEQ ID NO: 9 - DNA, 744 bp, Artificial Sequence
  Gene in plasmid pTrc-EcMt2ORF02331 codon-optimized for expression in E. coli (SEQ ID NO. 9)
atgtgcaaagtgcacctgtttaatgatgattgcctgaacgtgctgaaaaagatcgaagataatagcatcgatctgatctttgcagatccgccttataatctgagcagcgaaaatgcactgaccacacgtgcaggtaaaccggttaaatgttataaaggcgagtgggataaaatcgacgatatctttgaatttaacctgcgctggattgaacagtgtgttcgtgttctgaaagaaaccggcaccatttggattagcggcaccctgcataatcatccgattattggcaccattctgaaacagttaggtctgtggattatcaacgatatcatttggttcaaaccgaatgcaacaccgctgctgagccgtaatcgttttgttccgagcaccgaactgatttgggttgcaagcaaaaacaaaaaatactactttgattatgaaatggcacgcaaactgaatggtggtaaacaaatgcgtaatctgtgggaaattccggcacagcgtcataaaacaccgcatccgaccgaaaaaccggaagcactgctggaacgtattattctgattggtagcaaagaaggtgacgttgttctggatccgtttatgggtagcggtacaaccggtgttgttgcaaaactgctgaaacgcaactttattggcattgaaattgatccggtgtatttcgagattgccaaaaaacgcatcgaagaagaaaaactgattcagcagacctttagcaacttcctgtaa

SEQ ID NO: 10 - protein, 247 aa, Artificial Sequence
  Translation product of SEQ ID NO. 9 (SEQ ID NO. 10)
MCKVHLFNDDCLNVLKKIEDNSIDLIFADPPYNLSSENALTTRAGKPVKCYKGEWDKIDDIFEFNLRWIEQCVRVLKETGTIWISGTLHNHPIIGTILKQLGLWIINDIIWFKPNATPLLSRNRFVPSTELIWVASKNKKYYFDYEMARKLNGGKQMRNLWEIPAQRHKTPHPTEKPEALLERIILIGSKEGDVVLDPFMGSGTTGVVAKLLKRNFIGIEIDPVYFEIAKKRIEEEKLIQQTFSNFL

SEQ ID NO: 11 - DNA, 744 bp, Artificial Sequence
  Gene from Caldicellulosiruptor sp. strain DIB 104C (SEQ ID NO. 11)
atgtgcaaagtacatttatttaatgatgattgtttgaatgttctaaaaaagatagaagacaatagtatagatctgatttttgctgatcctccttacaatttgtcttcagaaaatgctctaaccacgagagccggtaaaccagtaaaatgttataaaggcgaatgggataaaatagatgatatatttgagtttaatctaaggtggatcgagcaatgtgtcagagtacttaaagaaactggaactatttggatttctggaacattgcataatcatcctataattggaactattctgaagcagttgggtctctggattatcaatgacattatatggtttaaacctaatgcaactcctttactttcaagaaatagatttgttccatctacagaattgatctgggttgcaagtaaaaataaaaaatattattttgactatgagatggcacgaaagcttaatggaggcaaacagatgagaaacttatgggaaattcctgctcaaaggcataagactcctcatcctactgaaaaacctgaagcattgttagaaaggattattctaataggcagtaaagaaggggatgtggtcttagatccttttatgggctctggaacaactggcgttgtagctaaattgcttaaacgaaactttattggaattgaaattgatccagtatactttgagattgcaaaaaaacgtattgaggaagaaaagcttattcagcaaactttttcaaattttctttaa

SEQ ID NO: 12 - protein, 247 aa, Artificial Sequence; identical to SEQ ID NO: 10
  Translation product of SEQ ID NO. 11 (SEQ ID NO. 12)

SEQ ID NO: 13 - DNA, 1785 bp, Artificial Sequence
  Sequence of gene DOCILOBI_02329 encoding for restriction enzyme of type II restriction modification system (gene from Caldicellulosiruptor sp. strain DIB 104C) (SEQ ID NO. 13)
atgcgaaagccttggtctatttccacaactgttagaaaccccgaaagattaagaggatttttgcaagtgttatctgagtttgaaggaatgaattttgatgagaacgttcaaattcaatatcagatacgattaatacaatacaaactctacagacctatgaatttacctgaagatataaaacgtgagtttgatgaccccgcaattaaaaaaatagattacaaaaaagctaagaagatttttgatttacaaaaatataaagatccttctatgcgtggtcggcagtctgtaaatcccttgaacaaattaggatttgcaattgccaaaaaaagcttaggcaagattagaataactgagttgggaagaaaatttttagatgaaaataacgatgtctccgagatacttttcaaaagtcttcttaaattacagtacccaaatccttttagtagcgattttaaaagcagagatggctttgatatcattccttttatagtaactttgaagtttttctatttgttagaaaaacaaacgcaaatagaagaaatttcaaaaagagaattttgtttattcattccaaccctcattaactataaagaaatagaaaatcaaattaaccagctacttgattatagaagatcaaaaaataagagagattttgaagtacatttcgtctctaagttttttggttcagctaaaaatattgagactaaaatcaacaatttgtttgattatggagataacatcttacgattttttcttcttacaaaatgttttaccgctaaaaaaagtgaatttggacaagttgccagtgtaaaactttcacaagatagaaaaaaggaaatcgaagaattactaaatatgtttgaaggaaaagcaataagcttttctaatttagatgactacatcgaatatatgactgatatcacaaaacctgaattgccgtgggaaaaaaataagaataaacttattgagatggcagaaagtatccggaaagatatttcacagcaaatagaaagcagtaaaattgcaataaatgaatactctaaaatggttttaggaaaaaacttatttgagttatctgaggaagaattgaaaaaacatatcaatgaactgagggcgataaaatccaaaattaacgaagctaaaaaggctcatatcttaaaatataactttgctaaattagacgaacatattaaaatactaaggaataaagaatattggaaggaattagaaccagccgatttagaacaaataatattcgagctattgttaattattgatagtgcagaaaaaattgaatcaaatgctattaaagatgatgaaggaaattttataaattttgcaccagctaaaaaacctgatattgaatttttcttcaaggaatttgctggaatttgtgaagtaacactaaacaaaactcaatatcaatggattcaagagggatatcctgtattagatcacgtggcaaaatttatgaatcaataccctaattatactaattttgtaaatatttttattgctccaaaaatacatgataatacctattataattttttcattgctttaaaatacggatttaagagtaagaaaatcagaattatccctttaaattttgaacagtttactatgttcacaaaagtgcttcaaagctattttgaaaagttcaatggtttaaattcaaacttaattttgaccctttgtaatgatatattttacacaatggaaaatttagatgaccactcaatgatatgtagtttaattgacaataagctttatagtctattttag

SEQ ID NO: 14 - DNA, 24 bp, Artificial Sequence
  primers MCUP180
agatcaaaggatcttcttgagatc

SEQ ID NO: 15 - DNA, 31 bp, Artificial Sequence
  BLU32
aagaaatagcggtctgacgctcagtggaacg

SEQ ID NO: 16 - DNA, 30 bp, Artificial Sequence
  primer 054_pyrEup_f
cttgtccgaacgtgaaagaaggtggaatgg

SEQ ID NO: 17 - DNA, 30 bp, Artificial Sequence
  055_pyrEdw_r
ttggcatttctcacgtgccagaaggaagac

SEQ ID NO: 18 - DNA, 30 bp, Artificial Sequence
  primer BLU001
aggtggactttcaggccctgctataaagcc

SEQ ID NO: 19 - DNA, 30 bp, Artificial Sequence
  BLU002
aacttgcagggacactttctggcggagaac

SEQ ID NO: 20 - protein, 25 aa, Caldicellulosiruptor kristjanssonii
NRITFSGTVDSGGFIFLDPPYYSTT

SEQ ID NO: 21 - protein, 25 aa, Caldicellulosiruptor kristjanssonii
NRITFSGTGDSGGFIFLDPPYYSTT

SEQ ID NO: 22 - protein, 25 aa, Caldicellulosiruptor lactoaceticus
NRITFSGTVDSGGFIFLDPPYYSTT

SEQ ID NO: 23 - protein, 25 aa, Anabaena variabilis
NRITFSGTVESGGFIFLDPPYFSAT

SEQ ID NO: 24 - protein, 230 aa, Artificial Sequence
  M.CbeI_GGCC_279aa
VGGGAVALAVKQLYTNVKIKINDLNYDLICFWKQLRDNPVQLIEEVSKIKENYKDGRKLYEFLTSQNGGGEFERAVRFYILNRITFSGTVDSGGYSQQSFENRFTWSAINKLKQAAEIIKDFEISHGDYEKLLFEPGNEVFIFLDPPYYSTTESRLYGKNGDLHLSFDHERFAFNIKKCPHLWMITYDDSPEVRKLFKFANIYEWELQYGMNNYKQSKAEKGKELFITNY

SEQ ID NO: 25 - protein, 240 aa, Caldicellulosiruptor sp. BluCon085
LGGGALLIELYNKGILKNAVVSDINLELINLYTAIKNCPDEVVYYIKNLDFKNAEDDYYKARELYNSIKIKNMGTIENENLLKAVLLLYLNRHCYNGLYRVNSKGEFNVPFGRYKNPKMPTSEEIFAFSEMLQSVEILHADFEEAVKKVSEFDFVYFDPPYMPVSKTANFTDYTVAGFSKVEQVRLKNVCDNLSKKGCFVMISNSDSEFIRDLYRNYNIEVIEAKRLINSTAEKRTGHKE

SEQ ID NO: 26 - DNA, 15 bp, Artificial Sequence
  methylated base pairs including the sequence motif 5?-GATGC-3?
catgcgatgccaggt

SEQ ID NO: 27 - DNA, 15 bp, Artificial Sequence
  methylated base pairs including the sequence motif 5?-GATGC-3? II
acaaagatgcatctc

SEQ ID NO: 28 - DNA, 15 bp, Artificial Sequence
  methylated base pairs including the sequence motif 5?-GATGC-3? III
tttatgatgcctgtg

SEQ ID NO: 29 - DNA, 15 bp, Artificial Sequence
  methylated base pairs including the sequence motif 5?-GCATC-3? I
ccaaagcatcttata

SEQ ID NO: 30 - DNA, 15 bp, Artificial Sequence
  methylated base pairs including the sequence motif 5?-GCATC-3? II
gcagcgcatcaccga

SEQ ID NO: 31 - DNA, 15 bp, Artificial Sequence
  methylated base pairs including the sequence motif 5?-GCATC-3? III
agagagcatctgcgt

SEQ ID NO: 32 - DNA, 14 bp, Artificial Sequence
  methylated base pairs including the sequence motif 5?- GATC-3? IV
ccgaagatcgggtt

SEQ ID NO: 33 - DNA, 14 bp, Artificial Sequence
  methylated base pairs including the sequence motif 5?- GATC-3? V
cagtagatcttgta

SEQ ID NO: 34 - DNA, 14 bp, Artificial Sequence
  methylated base pairs including the sequence motif 5?- GATC-3? VI
gcattgatcaggtt

SEQ ID NO: 35 - DNA, 81 bp, Artificial Sequence
  Reference pyrE-WT
tacctattgtaaaacctggaagtagaaaaagtaagtagatctaatttgcttgcataaatgattaaaattattaattccaaa

SEQ ID NO: 36 - DNA, 80 bp, Artificial Sequence
  Amplicon primer 054/055
tacctattgtaaaacctggaagtagaaaaagtaagtagattaatttgcttgcataaatgattaaaattattaattccaaa

SEQ ID NO: 37 - DNA, 81 bp, Artificial Sequence; identical to SEQ ID NO: 35
  pyrE-sequence clone #1

SEQ ID NO: 38 - DNA, 81 bp, Artificial Sequence; identical to SEQ ID NO: 35
  pyrE-sequence clone #2

SEQ ID NO: 39 - DNA, 81 bp, Artificial Sequence; identical to SEQ ID NO: 35
  pyrE-sequence clone #3